(19) United States
(12) Patent Application Publication
(10) Pub. No.: US 2010/0260671 A1
(43) Pub. Date: Oct. 14, 2010

(54) COMPOUNDS AND METHODS FOR THE DIAGNOSIS AND TREATMENT OF AMYLOID ASSOCIATED DISEASES

(75) Inventors: Jerry Yang, La Jolla, CA (US); Petra Inbar, Glen Rock, NJ (US); Mahealani Bautista, Orangevale, CA (US); Michael Mayer, Ann Arbor, MI (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF THE UNIVERSITY OF CALIFORNIA, OAKLAND, CA (US)

(21) Appl. No.: 12/601,229

(22) PCT Filed: May 30, 2008
(86) PCT No.: PCT/US08/65410

§ 371 Date: Jun. 30, 2010

Related U.S. Application Data
(60) Provisional application No. 60/940,869, filed on May 30, 2007.

(51) Int. Cl. A61K 51/04; C12N 5/071; A61K 31/465; A61K 31/137; A61K 31/12; A61K 31/60; A61K 31/195; A61K 31/235; A61K 31/40; A61P 25/28

(52) U.S. Cl. 424/1.65; 435/325; 514/343; 514/653; 514/679; 514/159; 514/567; 514/533; 514/408

(57) ABSTRACT

The invention is in general directed to compounds, such as tannic acid, nicotine, nicotine derivatives and pyrrolid derivatives of nicotine, and methods for diagnosing, preventing or alleviating the symptoms of amyloid-associated diseases, for example, neuronal diseases, such as, for example, Alzheimer's disease, compounds and methods for inhibiting ion channel activity of beta amyloid, and methods of diagnostic imaging of A/3 fibrils.

### CROSS REFERENCE TO RELATED APPLICATIONS

This application claims priority to U.S. Provisional Application Ser. No. 60/940,869 filed May 30, 2007. In addition, this application is related to U.S. patent application Ser. No. 11/487,224 filed Jul. 14, 2006, and to U.S. Provisional Application Ser. No. 60/932,293, filed May 30, 2007 by the University of Michigan. Accordingly, this application incorporates by reference in its entirety all subject matter of the above-referenced applications to the extent such subject matter is not inconsistent herewith.

### TECHNICAL FIELD

The invention is in general directed to compounds, such as tannic acid, nicotine, nicotine derivatives and pyrrolidine derivatives of nicotine, and methods for diagnosing, preventing or alleviating the symptoms of amyloid-associated diseases, for example, neuronal diseases, such as, for example, Alzheimer's disease, compounds and methods for inhibiting ion channel activity of beta amyloid, and methods of diagnostic imaging of Aβ fibrils.

### BACKGROUND

Amyloid fibrils formed from misfolded proteins or peptides are a hallmark of many neuronal diseases, such as, for example, Alzheimer's disease. (Soto, C. Nature Rev. Neurosci. 2003, 4: 49; Agorogiannis, E. I., et al., Neuro path. Appl. Neurobiol. 2004, 30:215; Kelly, J. W. Structure 1997, 5:595). Amyloid fibrils have also been associated with other, non-neuronal diseases and conditions, such as, for example, those listed in Table 2.

Amyloid fibrils and plagues are rich in beta sheet structure. Aβ is a peptide found in amyloid fibrils and plaques. Researchers have associated the development of Alzheimer's disease (AD), with the interaction of Aβ peptides, oligomers, and fibrils with cellular components in the brain. (Dawbarn, D., and Allen, S. J. Neurobiology of Alzheimer's disease, second ed., Oxford University Press, Oxford, 2001; Pereira, C., et al., J. Mol. Neurosci. 2004, 23: 97.) The interaction between cellular proteins, such as, for example, catalase, ABAD (beta amyloid-binding alcohol dehydrogenase) and RAGE (receptor for advanced glycation end products) and aggregated Aβ-amyloid fibrils (Aβ fibrils), for example, have been reported for their potential contribution to Aβ-induced neurotoxicity in the pathogenesis of AD. (Milton, N. G. N. Biochem. J. 1999, 344: 293-296; Milton, N. G. N., et al. Neuroreport 2001, 121: 2561; Yan, S. D., et al. Nature 1997, 389: 689; Yan, S. D., et al. J. Biol. Chem. 1999, 274: 2145; Lustbader, J. W., et al. Science 2004, 304: 448; Yen, S. D., et al. Nature 1996, 382: 685; Yan, S. D, et al., Am. J. Pathol. 1999, 155: 1403; Yan, S. D., et al., Biochim. Biophys. Acta 2000, 1502: 145; K. Takuma, J. Yao, J. Huang, H. Xu, X. Chen, J. Luddy, A.-C. Trillat, D. M. Stern, O. Arancio, S. S. Yan, FASEB J. 2005, 19(6), 597-598; Takuma, K., et al., FASEB J. 2005, 19(6): 597-598)

Several classes of small molecule therapeutics are used clinically to treat the symptoms of AD, such as, for example, inhibitors of cholinesterase. (Francis, P. T., et al., Trends Pharm. Sci. 2005, 26: 104; Conway, K. A., et al., Curr. Pharm. Design 2003, 9: 427.) Current strategies to modify directly the pathology of AD using synthetic molecules are focused mainly on slowing down the production of Aβ peptide or preventing the growth of Aβ fibrils. (C. Schmuck, et al., Chem Bio Chem 2005, 6: 1; C. N. Johnson, et al., Drug Dis. Today 2004, 1: 13; M. S. Parihar and T. Hemnani, J. Clin. Neurosci. 2004, 11: 456; V. M.-Y. Lee, Neurobio. Aging 2002, 23: 1039; B. Bohrmann, et al., J. Biol. Chem. 1999, 274: 15990; F. G. De Felice, et al., FASEB J. 2004, 18:1366; M. A. Findeis, Biochim. Biophys. Acta 2000, 1502:76.; J. E. Gestwicki, et al., Science 2004, 306: 865).

Other strategies focus on disrupting the fibrils so that they disassemble into their Aβ peptide components. These approaches may increase the amount of Aβ peptide, Aβ-dimers, or small Aβ oligomers in neurons, which may have a toxic affect.

In addition, evidence has been reported that Aβ oligomers and Aβ fibrils destabilize membrane potentials and disrupt calcium homeostasis in neurons through the formation of ion channels. (Lin, H., Bhatia, R. & Lal, R. Faseb J 15, 2433-44 (2001); Kourie, J. I., Henry, C. L. & Farrelly, P. Cell Mol Neurobiol 21, 255-84 (2001); Novarino, G., et al. J Neurosci 24, 5322-30 (2004); Plant, L. D. et al. Neurobiol Aging (2005); Ma, Z. G., Wang, J., Jiang, H., Xie, J. X. & Chen, L. Neurosci Lett 382, 102-5 (2005); Brown, S. T. at al. J Biol Chem 280, 21706-12 (2005); Lessard, C. B., Lussier, M. P., Cayouette, S., Bourque, G. & Boulay, G. Cell Signal 17, 437-45 (2005); Ronquist, G. & Waldenstrom, A. J Intern Med 254, 517-26 (2003); Rogawski, M. A. & Wenk, G. L. CNS Drug Rev 9, 275-308 (2003); Plant, L. D. et al. Neuroreport 13, 1553-6 (2002); Kagan, B. L., Hirakura, Y., Azimov, R., Azimova, R. & Lin, M. C. Peptides 23, 1311-5 (2002); Lin, M. C. & Kagan, B. L. Peptides 23, 1215-28 (2002); Suh, Y. H. et al. J Neural Transm Suppl, 65-82 (2000); Pettit, D. L., Shao, Z. & Yakel, J. L. J Neurosci 21, RC120 (2001); Ma, Z. G., Wang, J., Jiang, H., Xie, J. X. & Chen, L. Neurosci Lett 382, 102-5 (2005); Lessard, C. B., Lussier, M. P., Cayouette, S., Bourque, G. & Boulay, G. Cell Signal 17, 437-45 (2005); Quist, A. et al. Proc. Nat. Acad. Sci. USA 102, 10427-10432 (2005); Mucke, L. at al. J. Neurosci. 20, 4050-4058 (2000); Hsia, A. Y. et al. Proc. Nat. Acad. Sci. USA 96, 3228-3233 (1999); Rhee, S. K., Quist, A. P. & Lal, R. J. Biol. Chem. 273, 13379-13382 (1998); Lambert, M. P. et al. Proc. Nat. Acad. Sci. USA 95, 6448-6453 (1998); Hartley, D. M. et al. Journal of Neuroscience 19, 8876-8884 (1999). Mattson, M. P., Begley, J. G., Mark, R. J. & Furukawa, K. Brain Res 771, 147-53 (1997).) Several groups have reported that non-fibrillar forms of aggregated A-beta are sufficient to induce toxicity in neurons. (Hartley, D. M. at al. Journal of Neuroscience 19, 8876-8884 (1999); Quist, A. et al. Proc. Nat. Acad. Sci. USA 102, 10427-10432 (2005); Mucke, L. et al. J. Neurosci. 20, 4050-4058 (2000); Hsia, A. Y. et al. Proc. Nat. Acad. Sci. USA 96, 3228-3233 (1999); Rhee, S. K., Quist, A. P. & Lal, R. J. Biol. Chem. 273, 13379-13382 (1998); Lambert, M. P. et al. Proc. Nat. Acad. Sci. USA 95, 6448-6453 (1998)) One of the most prevalent hypotheses for the deleterious effects of oligomers of Aβ peptides is based on the generation of pore-like structures in cellular membranes that lead to acute electrophysiological changes and neuronal dysfunction in AD (solutions of A-beta fibrils have also been found to induce ion channel activity) (Hartley, D. M. et al. Journal of Neuroscience 19, 8876-8884 (1999); Lin, H., Bhatia, R. & Lal, R. Faseb J 15, 2433-44 (2001); Novarino, G. at al. J Neurosci 24, 5322-30 (2004).)

On more commonly studied ion channel-forming peptides than A-beta (e.g. on antibiotic peptides such as alamethicin) it has been reported that the binding of molecules to these peptides can inhibit the ion channel activity through disruption of the pores. (Lougheed, T., Zhang, Z. H., Woolley, G. A. & Borisenko, V. Bioorg. Med. Chem. 12, 1337-1342 (2004); Borisenko, V., Zhang, Z. H. & Woolley, G. A. Biochim. Biophys. Acta-Biomembr. 1558, 26-33 (2002); Cornell, B. A. et al. Nature 387, 580-583 (1997); Futaki, S. et al. Bioorg. Med. Chem. 12, 1343-1350 (2004); Terrettaz, S., Ulrich, W. P., Guerrini, R., Verdini, A. & Vogel, H. Angew. Chem.-Int. Edit. 40, 1740-1743 (2001); Lougheed, T., Borisenko, V., Hennig, T., Ruck-Braun, K. & Woolley, G. A. Org. Biomol. Chem. 2, 2798-2801 (2004); Clark, T. D., Buehler, L. K. & Ghadiri, M. R. J. Am. Chem. Soc. 120, 651-656 (1998); Bali, D., King, L. & Kim, S. Aust. J. Chem. 56, 293-300 (2003).)

Accordingly, there is a need for novel methods and compounds for diagnosing and treating amyloid-associated diseases, for example, neuronal diseases and conditions, with a smaller incidence of toxicity.

### SUMMARY

Provided herein are compounds and methods for preventing or alleviating the symptoms of amyloid-associated diseases, for example, but not limited to, neuronal diseases and conditions associated with amyloid fibril or plaque formation. It has been found that providing a binding molecule that coats the surface of an Aβ fibril may allow the fibrils to resist the interaction of cellular proteins with these fibrils, resulting in a new strategy to intervene in AD-related pathology. Provided herein are compounds and methods for inhibiting the binding interaction between Aβ fibrils and cellular proteins. Provided herein are compounds and methods for inhibiting or disrupting the ion channel activity of beta amyloid. Provided also are compounds and methods used for diagnoses or prognoses of amyloid associated diseases, for example.

In one embodiment, a method of inhibiting or disrupting Aβ fibril interaction with cellular proteins is provided. The method includes contacting the Aβ fibril with a compound selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or derivatives and analogs thereof. In general the cellular protein is expressed in neural tissue such as brain tissue. In some aspects, the Aβ fibril interaction with cellular proteins is associated with a neuronal disease such as Alzheimer's disease, Parkinson's disease, Huntington's disease, Down's Syndrome, cerebrovascular amyloidosis, Lewy body dementia, or spongiform encephalopathy.

In another embodiment, a method of inhibiting or disrupting ion channel activity of beta amyloids associated with a neuronal disease, comprising contacting a beta amyloid with a compound selected from the group consisting of tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, and BTA-EG₆.

In some embodiments, the compound is nornicotine, 5-bromonornicotine, 5-bromonicotine, and 5-iodonicotine. In other embodiments the compound is a nicotinic ester, a 5-bromopicolinic ester, and a picolinic ester.

In another embodiment, a method of preventing or alleviating the symptoms of an amyloid-associated neuronal disease is provided. The method includes contacting a subject with a compound selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or derivative or analogs thereof. In some aspects, the compound inhibits or disrupts Aβ fibril interactions with cellular proteins.

In yet another embodiment, a method for diagnosing an amyloid associated disease in a subject is provided. The method includes administering an Aβ fibril-binding compound to an individual and detecting the binding of the compound to amyloid deposits in the individual, wherein the compound is selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or any combination thereof.

In another embodiment, a method for detecting amyloid deposits in a subject is provided. The method includes administering a pharmaceutical composition comprising a pharmaceutically acceptable carrier and a compound selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or any combination thereof; and detecting the binding of the compound to an amyloid deposit in the subject.

In another embodiment, a method of preventing or alleviating the symptoms of an amyloid associated disease is provided. The method includes contacting Aβ fibrils with a sufficient amount of a pharmaceutical composition comprising a pharmaceutically acceptable carrier and an Aβ fibril binding compound selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or any combination thereof, wherein the interactions of the Aβ fibrils with a second binding molecule are inhibited. In some aspects, the Aβ fibril-binding compound is detectably labeled with, for example, a radio-label.

In another embodiment, a method of preventing or alleviating the symptoms of an amyloid associated disease is provided. The method includes contacting Aβ fibrils with a sufficient amount of a pharmaceutical composition comprising a pharmaceutically acceptable carrier and an Aβ fibril binding compound selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or any combination thereof, wherein the ion channel activity of the Aβ fibril decreases.

In another embodiment, a composition that includes a compound bound to one or more Aβ fibrils. In general the compound can be tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, ETA-EG₄, or BTA-EG₆, or any combination thereof.

In another embodiment, a pharmaceutical composition comprising a compound suitable for treating a neuronal disease is provided. In general, the composition includes a compound such as tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or any combination thereof. In one aspect, the compound inhibits or disrupts Aβ fibril interactions with cellular proteins.

In another embodiment a compound having a formula or structure of a compound listed in Table 1 or Table 5 is provided.

### BRIEF DESCRIPTION OF THE DRAWINGS

FIG. 1. Illustration of the inhibition of binding of Aβ-binding proteins to Aβ fibrils using small molecules. a) In this cartoon, the small molecules compete with the Aβ-binding proteins for binding to the Aβ fibril (see Puchtler, H., et al., J. Histochem. Cytochem. 1962, 10: 355; LeVine III., H. Meth. Fnzym. 1999, 309: 274); b) chemical structures of Thioflavin T (ThT) and two derivatives of 2-(4-aminophenyl)-benzothiazoles (BTA-EG₄ and BTA-EG₆).

FIG. 2. Inhibition (Inhib.) of IgG-Aβ interactions with ThT. a) Aβ fibrils incubated with solutions of ThT and exposed to an anti-Aβ IgG (clone 6E10). b) Same assay as in (a) but using an anti-Aβ IgG raised against a different binding epitope of Aβ peptide (clone AMY-33). c) Same assay as in (a) except the inhibition is plotted against the concentration of 1-naphthol-4-sulfonate (NS) instead of ThT.

FIG. 3. Inhibition (Inhib.) of IgG-Aβ interactions as a function of increasing concentrations (conc.) of ThT. ThT and the fibrils were incubated together prior to depositing the ThT-coated fibrils into 96-well plates and exposure to an anti-Aβ IgG (clone 6E10, derived from Aβ residues 3-8 as antigens).

FIG. 4. Disruption of Aβ ion channels by addition of nicotine at increasing concentrations from A to C. Addition of nicotine at 4-fold molar excess with respect to Aβ disrupted preformed ion channels almost completely within 10 minutes.

FIG. 5. Inhibition of Aβ ion channel formation by nicotine in a molar ratio to Aβ of 1:1, A) Addition of 37 μM Aβ in the absence of nicotine resulted in ion channel activity in less than 30 minutes. B) Addition of the same Aβ concentration together with 37 μM nicotine did not result in ion channel activity during the entire test period (90 min). Note the different y-scales in A and B.

FIG. 6. Inhibition of Aβ ion channel formation by tannic acid in a molar ratio to Aβ of 1:1.

FIG. 7. Example of a device for measuring ion channel inhibition.

FIG. 8. Ability of nicotine derivatives to coat Aβ fibrils: A) nicotine; B) 5-Bromonicotine; C) 5-Bromonicotinic ester; D) N-tetraethylene glycol nicotine.

FIG. 9. Exemplary data from lipid bilayer experiments are shown. A current baseline at voltage −50 mV was applied. Results in the presence of Aβ(1-42) (2 μM) and Aβ(1-42) in the presence of N-Me Dopamine at final concentration of 450 μM after observing Aβ ion channel activity are shown.

FIG. 10. Exemplary data from lipid bilayer experiments are shown. A current baseline at voltage −50 mV was applied. Results in the presence of Aβ(1-42) (11 μM) and Aβ(1-42) pre-incubated with BTA-EG₆ (1:20 molar ratio; final [Aβ(1-42)]=11 μM; final [BTA-EG₆]=220 μM) are shown.

### DETAILED DESCRIPTION

Provided herein are compounds, such as tannic acid, nicotine, nicotine derivatives and pyrrolidine derivatives of nicotine, and methods for diagnosing, preventing or alleviating the symptoms of amyloid-associated diseases, for example, neuronal diseases, such as, for example, Alzheimer's disease, compounds and methods for inhibiting ion channel activity of beta amyloid, and methods of diagnostic imaging of Aβ fibrils.

Table 1 provides a list of exemplary compounds useful for treating amyloid associated disorders:

### Halogenated and Oligoethylene Glycol Derivatives of Nicotine

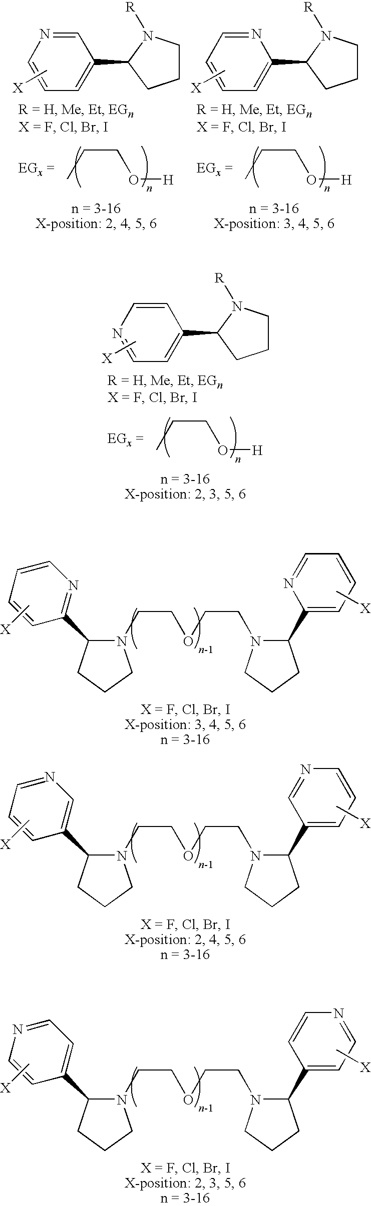

### Pyrrodidline Derivatives

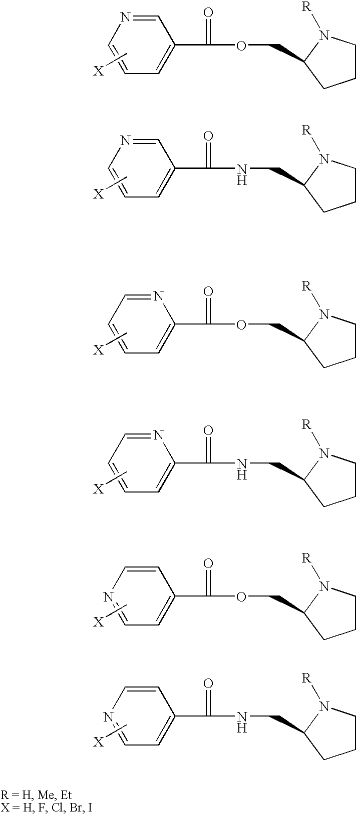

Compounds provided herein further include molecules 1-10 listed in Table 5 and their associated structures.

In exemplary embodiments of the invention are provided pyrrolidine derivatives of nicotine, as provided in Table 1.

The present invention further provides pharmaceutical compositions comprising a pharmaceutically acceptable excipient and a compound of Table 1 or Table 5. In exemplary embodiments, the compound of Table 1 is selected from the group consisting of pyrrolidine derivatives of nicotine, such as, for example, those provided in Table 1 and Table 5.

Also provided in the present invention is a method of preventing or alleviating the symptoms of an amyloid-associated disease comprising contacting Aβ fibrils with a compound selected from the group consisting of tannic acid, nicotine, nicotine derivatives of Table 1, and pyrrolidine derivatives of Table 1 and molecules set forth in Table 5. In exemplary embodiments, the compound is a pyrrolidine derivative of Table 1 or a molecule set forth in Table 5. The compound may be, for example, a nicotinic ester, a 5-bromopicolinic ester, or a picolinic ester of nicotine, as set out in Table 1. In exemplary embodiments, the disease is a neuronal disease. In further exemplary embodiments, the neuronal disease is selected from the group consisting of Alzheimer's disease, Parkinson's disease, Huntington's disease Down's Syndrome, and spongiform encephalopathy. For example, the neuronal disease may be, but is not limited to, Alzheimer's disease. Or, for example, the neuronal disease may be, but is not limited to, Parkinson's disease, Huntington's disease, Down's Syndrome, cerebrovascular amyloidosis, Lewy body dementia, or spongiform encephalopathy.

Also provided are methods of preventing or alleviating the symptoms of an amyloid-associated disease including contacting Aβ fibrils with a sufficient amount of a first binding molecule to decrease the interactions of the Aβ fibrils with a second binding molecule. In certain embodiments, the disease is a neuronal disease. In certain embodiments, a plurality of the first binding molecules forms an ordered layer on top of the fibrils. For example, the first binding molecule may coat a portion of the surface of the fibrils. The first binding molecule may, for example, coat more than 50, 55, 60, 65, 70, 75, 80, 85, 90, or 95% of the surface of the fibrils. The second binding molecule may be, for example a cellular component in the brain. The second binding molecule may be, for example, a cellular protein. The second binding molecule may be, for example, selected from the group consisting of catalase, ABAD, and RAGE. In certain embodiments of the present invention, the binding of the second binding molecule to the fibrils is associated with the symptoms of an amyloid associated disease, such as, for example, those listed in Table 1 or Table 5. In other embodiments of the present invention, the binding of the second binding molecule to the fibrils is associated with the symptoms of a neuronal disease, such as, for example, Alzheimer's disease, Parkinson's disease, Huntington's disease Down's Syndrome, or spongiform encephalopathy. In exemplary embodiments, the neuronal disease is Alzheimer's disease. In other exemplary embodiments, the neuronal disease is Parkinson's disease.

In some aspects of the present invention, the first binding molecule binds to the fibrils using hydrophobic and electrostatic interactions. In other aspects, the first binding molecule binds to the fibrils using non-covalent interactions with the fibrils. In certain aspects of the invention, the first binding molecule is selected from the group consisting of tannic acid, a derivative of tannic acid, or a nicotine derivative, such as, for example, nornicotine, 5-bromonornicotine, 5-bromonicotine, 5-iodonicotine, or a pyrrolidine derivative of nicotine such as, for example, those listed in Table 1 or Table 5. In exemplary embodiments, the compound is a pyrrolidine derivative of Table 1 or a derivative of a molecule set forth in Table 5. In other aspects of the invention, the first binding molecule is a compound of the present invention. In further embodiments of the invention, the method comprises administering a therapeutically effective amount of the first binding molecule to a subject or individual. By “individual” is meant, for example, any animal, for example, any mammal, such as, for example, a bovine, rodent, primate, horse, canine, feline, or human. In exemplary embodiments, the individual is human.

Also provided in the present invention is a method of inhibiting or disrupting Aβ fibril interaction with cellular proteins by contacting the Aβ fibril with a compound of the present invention. By inhibiting or disrupting is meant that decreased binding or interaction with cellular proteins is observed in the presence of the compound than in the absence of the compound, as measured using assays known to those of ordinary skill in the art, or as presented in the present application.

Also provided in the present invention is a method of inhibiting or disrupting ion channel activity of beta amyloids, comprising contacting a beta amyloid with a compound of the present invention. By inhibiting or disrupting is meant that decreased ion channel activity is measured in the presence of the compound when compared to ion channel activity in the absence of the compound, as measured using a method known to those of ordinary skill in the art, or by one of the assays presented in the present application. In certain aspects of the invention, the compound is selected from the group consisting of tannic acid, a derivative of tannic acid, nicotine, or a nicotine derivative, such as, for example, nornicotine, 5-bromonornicotine, 5-bromonicotine, 5-iodonicotine, or a pyrrolidine derivative of nicotine such as, for example, those listed in Table 1 or the molecules listed in Table 5. In exemplary embodiments, the compound is a pyrrolidine derivative of Table 1. In exemplary embodiments, the beta amyloids are associated with a neuronal disease. For example, the neuronal disease may be selected from the group consisting of Alzheimer's disease, Parkinson's disease, Huntington's disease Down's Syndrome, and spongiform encephalopathy. For example, the neuronal disease may be, but is not limited to, Alzheimer's disease. Or, for example, the neuronal disease may be, but is not limited to, Parkinson's disease.

The compounds of the present invention may also be used for diagnostic imaging of Aβ fibrils.

In yet other embodiments of the present invention are provided methods for diagnosing an amyloid associated disease in an individual, comprising administering an Aβ fibril-binding compound to an individual and detecting the binding of the compound to amyloid deposits in the individual, wherein the compound is selected from the group consisting of a compound of the present invention. In certain aspects of the invention, the compound is selected from the group consisting of tannic acid, a derivative of tannic acid, nicotine, or a nicotine derivative, such as, for example, nornicotine, 5-bromonornicotine, 5-bromonicotine, 5-iodonicotine, or a pyrrolidine derivative of nicotine such as, for example, those listed in Table 1. In exemplary embodiments the compound is a pyrrolidine derivative of Table 1. In further embodiments of the present invention are provided methods for identifying a change in the progress of an amyloid associated disease in an individual, comprising

administering an Aβ fibril-binding compound of the present invention to an individual and conducting a first detecting procedure to detect the binding of the compound to amyloid deposits in the individual on a first date;

administering an Aβ fibril binding compound of the present invention to the individual and conducting a second detecting procedure to detect the binding of the compound to amyloid deposits in the individual on a second date; and

comparing the amount, quantity, or other characteristics of the amyloid deposits detected in step b with the amyloid deposits detected in step a,

In yet further embodiments of the present invention are provided methods for detecting amyloid deposits in an individual, comprising

administering a pharmaceutical composition comprising a pharmaceutically acceptable carrier and a compound selected from the group consisting of a compound of the present invention. In certain aspects of the invention, the compound is selected from the group consisting of tannic acid, a derivative of tannic acid, or a nicotine derivative, such as, for example, nornicotine, 5-bromonornicotine, 5-bromonicotine, 5-iodonicotine, or a pyrrolidine derivative of nicotine such as, for example, those listed in Table 1 or a molecule listed in Table 5; and derivatives and analogs thereof; and

detecting the binding of the compound to an amyloid deposit in the individual. In exemplary embodiments, the pharmaceutical composition comprises a compound of the present invention. In certain embodiments, the amyloid deposit is present in the brain of the individual.

For detecting the presence of amyloid deposits, for example, the Aβ fibril-binding compound may be, for example, radiolabeled. Detection may be conducted by a method, for example, selected from the group consisting of gamma imaging, magnetic resonance imaging, or magnetic resonance spectroscopy. The detection may be, for example, single photon emission computed tomography or positron emission tomography.

In other embodiments of the present invention are provided methods for preventing or alleviating the symptoms of an amyloid associated disease comprising contacting Aβ fibrils with a sufficient amount of a pharmaceutical composition comprising a pharmaceutically acceptable carrier and an Aβ fibril binding compound selected from the group consisting of a compound of the present invention, such as, for example, tannic acid, a derivative of tannic acid, or a nicotine derivative, such as, for example, nornicotine, 5-bromonornicotine, 5-bromonicotine, 5-iodonicotine, or a pyrrolidine derivative of nicotine such as, for example, those listed in Table 1, and derivatives and analogs thereof, to decrease the interactions of the Aβ fibrils with a second binding molecule. In exemplary embodiments, the disease is a neuronal disease. In exemplary embodiments, the pharmaceutical composition comprises a compound of the present invention.

In other embodiments of the present invention are provided methods for preventing or alleviating the symptoms of an amyloid associated disease in an individual comprising administering to the individual a therapeutically effective dose of a pharmaceutical composition comprising a pharmaceutically acceptable carrier and an Aβ fibril binding protein selected from the group consisting of nornicotine, 5-bromonornicotine, 5-bromonicotine, 5-iodonicotine and a pyrrolidine derivative of nicotine such as, for example, those listed in Table 1 or a molecule listed in Table 5, and derivatives and analogs thereof, in a pharmaceutically acceptable carrier. In exemplary embodiments, the disease is a neuronal disease. In exemplary embodiments, the pharmaceutical composition comprises a compound of the present invention. The neuronal disease may be, for example, Alzheimer's disease; the neuronal disease may be, for example, Parkinson's disease.

Also provided in the present invention are reagents that include a compound of the present invention. In certain aspects of the invention, the compound is selected from the group consisting of tannic acid, a derivative of tannic acid, or a nicotine derivative, such as, for example, nornicotine, 5-bromonornicotine, 5-bromonicotine, 5-iodonicotine, or a pyrrolidine derivative of nicotine such as, for example, those listed in Table 1 or those listed in Table 5. The research reagent may be, for example, formulated to detect amyloid proteins in vivo. The research reagent may be, for example, formulated to detect amyloid proteins in cells or tissue, wherein the cells or tissue have been isolated from a living organism. Also provided in the present invention is a kit comprising a research reagent of the present invention

Diseases

By “amyloid associated diseases” is meant any disease or condition that is associated with the increased or decreased presence of amyloid proteins, such as the presence of amyloid plaques. The methods of the present invention may be used to diagnose or to detect a propensity for an amyloid-associated disease where no plaques are detected, such as, for example, by detecting amyloid protein as a biomarker. For example, the presence of amylin may be detected using the methods of the present invention, and this may be associated, for example, with a likelihood of developing type-two diabetes. Examples of amyloid associated diseases may be found in, but are not limited to, for example, Table 2.

Neuronal diseases that may be diagnosed, treated, prevented or exhibit an alleviation of symptoms according to the present invention include any neuronal disease or condition, including, for example, neurodegenerative diseases, in which Aβ peptides, oligomers, fibrils, or plaques are implicated, for example, but not limited to, Alzheimer's disease, Parkinson's disease, Huntington's disease, Down's Syndrome, and spongiform encephalopathies such as, for example, Bovine Spongiform Encephalopathy (mad cow disease), Kuru, Creutzfeldt-Jakob disease, and Fatal Familial Insomnia.

Exemplary diseases treatable and diagnosable by compositions and methods provided herein include those listed in the following Table 2:

| Amyloid | Disease | Reference |
|---|---|---|
| Amylin | Type 2 Diabetes | Goldsbury, C. S., et al., J |
| | | Struc. Biol. 1997, 119(1), 17-27; |
| | | Goldsbury, C., et al., J. |
| | | Struc. Biol. 2000, 130(2-3), |
| | | 352-362; Jimenez, J. L., et al., |
| | | Proc. Natl. Acad. Sci. 2002, |
| | | 99(14), 9196-9201. |
| Insulin | Type 2 Diabetes | Sipe, J. D., Ann. Rev. Biochem. |
| | | 1992, 61, 947-975 |
| Immunoglobin light chains | AL amyloidosis (liver) | Bellotti, V., et al., J. Struc. |
| | | Biol. 2000, 130(2-3), 280-289; |
| | | Sipe, J. D., Ann. Rev. Biochem. |
| | | 1992, 61, 947-975 |
| Amyloid A (Lipoprotein) | Reactive systemic | Sipe, J. D., Ann. Rev. Biochem. |
| | amyloidosis | 1992, 61, 947-975 |
| Transthyretin | senile systemic amyloidosis | Sipe, J. D., Ann. Rev. Biochem. |
| | (SSA), familial amyloid | 1992, 61, 947-975; Brito, |
| | polyneuropathy (FAP) and | R. M. M. et al., Curr. Med. |
| | familial amyloid | Chem. Immun. Endoc. Metab. |
| | cardiomyopathy (FAC) | Agents 2003, 3(4), 349-360; |
| | | Damas, A. M. and Saraiva, |
| | | M. J., J. Struc. Biol. 2000, |
| | | 130(2-3), 290-299; Buxbaum, |
| | | J. N., Curr. Opin. Rheumatol. |
| | | 2003, 16(1), 67-75. |
| β2 microglobulin | Dialysis, renal failure | Buxbaum, J. N., Curr. Opin. |
| | | Rheumatol. 2003, 16(1), 67-75. |
| Apolipoprotein A1 | Coronary heart disease, | Buxbaum, J. N., Curr. Opin. |
| | atherosclerosis | Rheumatol. 2003, 16(1), 67-75. |
| PrPSc | (Prion disease, sheep) | Wille, H., et al., J. Struc. |
| | | Biol. 2000, 130(2-3), 323-338 |
| α-synuclein | Parkinson's, Alzheimer's | El-Agnaf, O. M. A. and Irvine, |
| | | G. B., J. Struc. Biol. 2000, |
| | | 130(2-3), 300-309 |
| Cystatin C | Cerebral hemorrhage | Sipe, J. D., Ann. Rev. |
| | | Biochem. 1992, 61, 947-975 |

Methods

Compounds that may be used in the methods of the present invention include compounds found to bind to Aβ fibrils that prevent other cellular components from binding to the fibrils. Compounds that may be used in the methods of the present invention may, for example, have one or more of the following characteristics: low molecular weight, known and favorable pharmacokinetic properties, and known permeability across the blood-brain barrier.

Compounds that may be used in the methods of the present invention may include, for example, compounds of the present invention, including, for example, those listed in the embodiments presented herein.

Compounds that may be used in the methods of the present invention may be radiolabeled, for example, for diagnostic imaging, such as that performed using single photon emission computed tomography (SPECT) or positron emission tomography (PET). In illustrative embodiments, the compounds have the ability to cross the blood brain barrier. (Di, L., et al., Curr. Opin. Chem. Bio. 2003, 7(3), 402-408; Abraham, M. H., Eur. J. Med. Chem. 2004, 39(3), 235-240; Mathis, C. A., et al., Current Pharm. Design, 2004, 10:1469-92) Compounds that may be used in the methods of the present invention include, for example, those showing inhibitory activity for IgG-Aβ interactions, or inhibitory activity for ion channel activity.

In other examples of the present invention, compounds that may be used to prevent or alleviate the symptoms of a neuronal disease such as, for example, Alzheimer's disease include, for example, tannic acid, nicotine, nicotine derivatives and pyrrolidine derivatives of nicotine, such as, for example, those listed in Table 1. Compounds that may be used in the methods of the present invention include, for example, those showing inhibitory activity for IgG-Aβ interactions, or inhibitory activity for ion channel activity.

In yet other, illustrative examples of the present invention, compounds that may be used for diagnostic imaging of Aβ fibrils include, for example, tannic acid, nicotine, nicotine derivatives and pyrrolidine derivatives of nicotine, such as, for example, those listed in Table 1. Compounds that may be used in the methods of the present invention include, for example, those showing inhibitory activity for IgG-Aβ interactions, or inhibitory activity for ion channel activity.

Compounds

Compounds of the present invention include, for example, tannic acid, nicotine, nicotine derivatives and pyrrolidine derivatives of nicotine, such as, for example, those listed in Table 1. In exemplary embodiments, compounds of the present invention include, for example, the pyrrolidine derivatives of nicotine, such as nicotinic ester, 5-bromopicolinic ester, and picolinic ester derivatives of nicotine of Table 1.

The terms “halo” or “halogen,” by themselves or as part of another substituent, mean, unless otherwise stated, a fluorine, chlorine, bromine, or iodine atom.

The compounds of the present invention, and compounds used in the methods of the present invention, may exist as salts. The present invention includes such salts. Examples of applicable salt forms include hydrochlorides, hydrobromides, sulfates, methanesulfonates, nitrates, maleates, acetates, citrates, fumarates, tartrates (e.g., (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present invention and compounds used in the methods of the present invention, contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present invention and compounds used in the methods of the present invention, contain both basic and acidic functionalities that allow the compounds to be converted into either base or acid addition salts.

Certain compounds of the present invention, and compounds used in the methods of the present invention, can exist in unsolvated forms as well as solvated forms, including hydrated forms. In general, the solvated forms are equivalent to unsolvated forms and are encompassed within the scope of the present invention. Certain compounds of the present invention and compounds used in the methods of the present invention, may exist in multiple crystalline or amorphous forms. In general, all physical forms are equivalent for the uses contemplated by the present invention and are intended to be within the scope of the present invention.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C— or ¹⁴C-enriched carbon are within the scope of this invention.

The compounds of the present invention and compounds used in the methods of the present invention, may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, and compounds used in the methods of the present invention, whether radioactive or not, are encompassed within the scope of the present invention.

The compounds of the present invention may be synthesized using one or more protecting groups generally known in the art of chemical synthesis. The term “protecting group” refers to chemical moieties that block some or all reactive moieties of a compound and prevent such moieties from participating in chemical reactions until the protective group is removed, for example, those moieties listed and described in Greene, et al., Protective Groups in Organic Synthesis, 3rd ed. John Wiley & Sons (1999). It may be advantageous, where different protecting groups are employed, that each (different) protective group be removable by a different means. Protective groups that are cleaved under totally disparate reaction conditions allow differential removal of such protecting groups. For example, protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, without limitation, methyl, ethyl, and acetyl in the presence of amines blocked with acid labile groups such as t-butyl carbamate or with carbamates that are both acid and base stable but hydrolytically removable.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be blocked with oxidatively-removable protective groups such as 2,4-dimethoxybenzyl, while co-existing amino groups may be blocked with fluoride labile silyl carbamates.

Allyl blocking groups are useful in the presence of acid- and base-protecting groups since the former are stable and can be subsequently removed by metal or pi-acid catalysts. For example, an allyl-blocked carboxylic acid can be deprotected with a palladium(O)-catalyzed reaction in the presence of acid labile t-butyl carbamate or base-labile acetate amine protecting groups. Yet another form of protecting group is a resin to which a compound or intermediate may be attached. As long as the residue is attached to the resin, that functional group is blocked and cannot react. Once released from the resin, the functional group is available to react.

The term “pharmaceutically acceptable salts” is meant to include salts of active compounds which are prepared with relatively nontoxic acids or bases, depending on the particular substituent moieties found on the compounds described herein. When compounds of the present invention and compounds used in the methods of the present invention, contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention and compounds used in the methods of the present invention, contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

In addition to salt forms, the present invention provides compounds, which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Descriptions of compounds of the present invention are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions.

Assays

Provided herein in the Examples are examples of screening assays that utilize 96 well microtiter plates. It will be apparent to those of ordinary skill in the art that these assays may be adapted for other types of microtiter plates, including those made of various materials and comprising various numbers of wells. Further, it is apparent to those of ordinary skill in the art that these assays may be adapted to other high throughput methods, including other solid supports methods such as beads, microarrays, and stamping. (Mayer, M., et al., Proteomics, 2004, 4:2366-76; G. MacBeath and S. L. Schreiber, Science, 2000 289(5485): 1760-1763.)

The Aβ fibrils, Aβ fibrils pre-incubated with a test compound, or the detection reagent may, for example, be immobilized to a solid support. It is understood that immobilization can occur by any means, including for example; by covalent attachment, by electrostatic immobilization, by attachment through a ligand/ligand interaction, by contact or by depositing on the surface.

As used herein “solid support” or “solid carrier” means any solid phase material upon which an oligomer is synthesized, attached, ligated or otherwise immobilized. Solid support encompasses terms such as “resin”, “solid phase”, “surface” and “support”. A solid support may be composed of organic polymers such as polystyrene, polyethylene, polypropylene, polyfluoroethylene, polyethyleneoxy, and polyacrylamide, as well as co-polymers and grafts thereof. A solid support may also be inorganic, such as glass, silica, controlled-pore-glass (CPG), or reverse-phase silica. The configuration of a solid support may be in the form of beads, spheres, particles, granules, a gel, or a surface. Surfaces may be planar, substantially planar, or non-planar. Solid supports may be porous or non-porous, and may have swelling or non-swelling characteristics. A solid support may be configured in the form of a well, depression or other container, vessel, feature or location. A plurality of solid supports may be configured in an array at various locations, addressable for robotic delivery of reagents, or by detection means including scanning by laser illumination and confocal or deflective light gathering.

Microarray or array means a predetermined spatial arrangement of samples present on a solid support or in an arrangement of vessels. These samples may be, for example, Aβ fibrils, Aβ fibrils pre-incubated with test compounds, or may, for example, be second binding molecules or detection antibodies where, for example, the solid support is bound to the detection reagent, and the assay comprises adding the pre-incubated Aβ fibrils to the second binding molecule. Certain array formats are referred to as a “chip” or “biochip” (M. Schena, Ed. Microarray Biochip Technology, BioTechnique Books, Eaton Publishing, Natick, Mass. (2000). An array can comprise a low-density number of addressable locations, e.g. 2 to about 12, medium-density, e.g. about a hundred or more locations, or a high-density number, e.g. a thousand or more. Typically, the array format is a geometrically regular shape that allows for fabrication, handling, placement, stacking, reagent introduction, detection, and/or storage. The array may be configured in a row and column format, with regular spacing between each location. Alternatively, the locations may be bundled, mixed or homogeneously blended for equalized treatment or sampling. An array may comprise a plurality of addressable locations configured so that each location is spatially addressable for high-throughput handling, robotic delivery, masking, or sampling of reagents, or by detection means including scanning by laser illumination and confocal or deflective light gathering.

The presence of a compound that blocks the binding of a second binding molecule to Aβ fibrils is generally detected using a second binding molecule that binds to Aβ fibrils. The second binding molecule is either directly labeled, i.e., comprise or reacts to produce a detectable label, or is indirectly labeled, i.e., bind to a molecule comprising or reacting to produce a detectable label. Labels can be directly attached to or incorporated into the detection reagent by chemical or recombinant methods.

In one embodiment, the detection reagent, the molecule that is detected in the screening assay, is a second binding molecule that is an antibody that specifically binds to Aβ peptide. In another embodiment, the detection reagent is an antibody that specifically binds to the second binding molecule. In one embodiment, a label is coupled to the detection reagent through a chemical linker. Linker domains are typically polypeptide sequences, such as poly gly sequences of between about 5 and 200 amino acids. In some embodiments, proline residues are incorporated into the linker to prevent the formation of significant secondary structural elements by the linker. Preferred linkers are often flexible amino acid subsequences which are synthesized as part of a recombinant fusion protein comprising the RNA recognition domain. In one embodiment, the flexible linker is an amino acid subsequence that includes a praline, such as Gly(x)-Pro-Gly(x) where x is a number between about 3 and about 100. In other embodiments, a chemical linker is used to connect synthetically or recombinantly produced recognition and labeling domain subsequences. Such flexible linkers are known to persons of skill in the art. For example, polyethylene glycol) linkers are available from Shearwater Polymers, Inc. Huntsville, Ala. These linkers optionally have amide linkages, sulfhydryl linkages, or heterofunctional linkages.

The detectable labels used in the assays of the present invention, which are attached to the detection reagent, can be primary labels (where the label comprises an element that is detected directly or that produces a directly detectable element) or secondary labels (where the detected label binds to a primary label, e.g., as is common in immunological labeling). An introduction to labels, labeling procedures and detection of labels is found in Polak and Van Noorden (1997) Introduction to Immunocytochemistry, 2nd ed., Springer Verlag, N.Y. and in Haugland (1996) Handbook of Fluorescent Probes and Research Chemicals, a combined handbook and catalogue Published by Molecular Probes, Inc., Eugene, Oreg. Patents that described the use of such labels include U.S. Pat. Nos. 3,817,837; 3,850,752; 3,939,350; 3,996,345; 4,277,437; 4,275,149; and 4,366,241.

Primary and secondary labels can include undetected elements as well as detected elements. Useful primary and secondary labels in the present invention can include spectral labels such as green fluorescent protein, fluorescent dyes (e.g., fluorescein and derivatives such as fluorescein isothiocyanate (FITC) and Oregon Green™, rhodamine and derivatives (e.g., Texas red, tetrarhodimine isothiocynate (TRITC), etc.), digoxigenin, biotin, phycoerythrin, AMCA, CyDyes™, and the like), radiolabels (e.g. ³H, ¹²⁵I, ³⁵S, ¹⁴C, ³²P, ³³P, etc.), enzymes (e.g., horse radish peroxidase, alkaline phosphatase etc.), spectral calorimetric labels such as colloidal gold or colored glass or plastic (e.g. polystyrene, polypropylene, latex, etc.) beads. The label can be coupled directly or indirectly to a component of the detection assay (e.g., the detection reagent) according to methods well known in the art. As indicated above, a wide variety of labels may be used, with the choice of label depending on sensitivity required, ease of conjugation with the compound, stability requirements, available instrumentation, and disposal provisions.

Preferred labels include those that use: 1) chemiluminescence (using horseradish peroxidase and/or alkaline phosphatase with substrates that produce photons as breakdown products as described above) with kits being available, e.g., from Molecular Probes, Amersham, Boehringer-Mannheim, and Life Technologies/Gibco BRL; 2) color production (using both horseradish peroxidase and/or alkaline phosphatase with substrates that produce a colored precipitate (kits available from Life Technologies/Gibco BRL, and Boehringer-Mannheim)); 3) fluorescence using, e.g., an enzyme such as alkaline phosphatase, together with the substrate AttoPhos (Amersham) or other substrates that produce fluorescent products, 4) fluorescence (e.g., using Cy-5 (Amersham), fluorescein, and other fluorescent tags); 5) radioactivity. Other methods for labeling and detection will be readily apparent to one skilled in the art.

For use of the present invention in the clinic, preferred labels are non-radioactive and readily detected without the necessity of sophisticated instrumentation. Preferably, detection of the labels will yield a visible signal that is immediately discernable upon visual inspection. One preferred example of detectable secondary labeling strategies uses an antibody that recognizes Aβ amyloid fibrils in which the antibody is linked to an enzyme (typically by recombinant or covalent chemical bonding). The antibody is detected when the enzyme reacts with its substrate, producing a detectable product. Preferred enzymes that can be conjugated to detection reagents of the invention include, e.g., β-galactosidase, luciferase, horse radish peroxidase, and alkaline phosphatase. The chemiluminescent substrate for luciferase is luciferin. One embodiment of a fluorescent substrate for β-galactosidase is 4-methylumbelliferyl-β-D-galactoside. Embodiments of alkaline phosphatase substrates include p-nitrophenyl phosphate (pNPP), which is detected with a spectrophotometer; 5-bromo-4-chloro-3-indolyl phosphate/nitro blue tetrazolium (BCIP/NBT) and fast red/napthol AS-TR phosphate, which are detected visually; and 4-methoxy-4-(3-phosphonophenyl)spiro[1,2-dioxetane-3,2′-adamantane], which is detected with a luminometer. Embodiments of horse radish peroxidase substrates include 2,2′azino-bis(3-ethylbenzthiazoline-6 sulfonic acid) (ARTS), 5-aminosalicylic acid (5AS), o-dianisidine, and o-phenylenediamine (OPD), which are detected with a spectrophotometer; and 3,3,5,5′-tetramethylbenzidine (TMB), 3,3′diaminobenzidine (DAB), 3-amino-9-ethylcarbazole (AEC), and 4-chloro-1-naphthol (4C1N), which are detected visually. Other suitable substrates are known to those skilled in the art. The enzyme-substrate reaction and product detection are performed according to standard procedures known to those skilled in the art and kits for performing enzyme immunoassays are available as described above.

The presence of a label can be detected by inspection, or a detector which monitors a particular probe or probe combination is used to detect the detection reagent label. Typical detectors include spectrophotometers, phototubes and photodiodes, microscopes, scintillation counters, cameras, film and the like, as well as combinations thereof. Examples of suitable detectors are widely available from a variety of commercial sources known to persons of skill. Commonly, an optical image of a substrate comprising bound labeling moieties is digitized for subsequent computer analysis.

Research Reagents

The present invention is further directed to research reagents used to detect amyloid proteins and amyloid plaques. Such research reagents are compounds that bind to amyloid proteins, including, for example, but not limited to, the compounds of the present invention. Research reagents of the present invention may further comprise dyes or other detectable labels. Thus, research reagents of the present invention include, for example, compositions that comprise the compounds of the present invention, and compounds of the present invention.

The research reagents may be used, for example, to detect the presence of amyloid plaques in vivo, in tissues, in cells, and in tissue or cell extracts. The research reagents may be used, for example, to determine the existence of an amyloid-associated disease, or to assist in screening for compounds that may prevent or alleviate the symptoms of the disease. The research reagents may be used, for example, to inhibit the interaction of an amyloid protein with a second binding protein, thus enabling the study of a cellular or disease mechanism. In one exemplary embodiment, a method is provided for detecting the presence of an amyloid protein, or an amyloid plaque, comprising contacting the amyloid protein or amyloid plaque with a research reagent of the present invention, and detecting binding of the research reagent to the amyloid protein or amyloid plaque.

Formulation

While the compounds of the present invention will typically be used in therapy for human patients, they may also be used in veterinary medicine to treat similar or identical diseases. The compounds of the present invention and compounds used in the methods of the present invention, include geometric and optical isomers.

The compounds according to the invention are effective over a wide dosage range. For example, in the treatment of adult humans, dosages from 0.01 to 1000 mg, from 0.02 to 800 mg, from 0.05 to 700 mg, from 0.1 to 650 mg, from 0.2 to 600 mg, from 0.5 to 500 mg, from 0.5 to 300 mg, from 0.5 to 250 mg, 0.5 to 100 mg, from 1 to 100 mg, from 1 to 50 mg, and from 1 to 50 mg per day, from 5 to 40 mg per day are examples of dosages that may be used. One example of a dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20 th ed.) Lippincott, Williams & Wilkins (2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

In therapeutic and/or diagnostic applications, the compounds of the invention may be formulated for a variety of modes of administration, including systemic and topical or localized administration. Techniques and formulations generally may be found in Remington: The Science and Practice of Pharmacy (20th ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20th ed.) Lippincott, Williams & Wilkins (2000). Suitable routes may include oral, buccal, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intraperitoneal, intranasal, or intraocular injections.

In illustrative embodiments, for injection, such as, for example, intravenous delivery, the agents of the invention may be formulated in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art. Use of pharmaceutically acceptable carriers to formulate the compounds herein disclosed for the practice of the invention into dosages suitable for systemic administration, or for targeted administration, such as that targeted to the brain, is within the scope of the invention. With proper choice of carrier and suitable manufacturing practice, the compositions of the present invention, in particular, those formulated as solutions, may be administered parenterally, such as by intravenous injection. The compounds may be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising one or more buffers, excipients, salts, preservative, auxiliaries and the like which facilitate processing of the active compounds into preparations which may be used pharmaceutically. The preparations formulated for oral administration may be in the form of tablets, dragees, capsules, or solutions. Appropriate pharmaceutically acceptable carriers are known to those of ordinary skill in the art and may be found in, for example, Remington: The Science and Practice of Pharmacy (20th ed.) Lippincott, Williams & Wilkins (2000).

Pharmaceutical preparations for oral use may be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that may be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

Pharmaceutical compositions of the present invention are those that, in addition to specifically binding amyloid in vivo and capable of crossing the blood brain barrier, are also non-toxic at appropriate dosage levels and have a satisfactory duration of effect.

### Examples

### Example 1

Beta-Amyloid Binding Assay using Thioflavin T (ThT): Synthetic reagents may be obtained from Aldrich, Fisher Scientific, Alfa Aesar or Fluka, and were used as received. Water was filtered through a NANOPure Diamond™ water purification system from Barnstead (18.2 μΩ/cm). Aβ-peptide (1-42) was obtained from Biopeptide Co, LLC, San Diego, Calif.; 96-well plates from Nalge Nunc International, Rochester, N.Y.; catalase from human erythrocytes (Lot #B67459) from Calbiochem, San Diego, Calif.; IgGs from Abcam, Cambridge, Mass.; and bovine serum albumin (BSA, fraction V) from Omni Pur.

NMR spectra were obtained on a Varian 400 MHz spectrometer. Chemical shifts are reported in ppm relative to residual solvent. FT-IR spectra were obtained on a Nicholet MAGNA-IR 440 spectrometer. A Perkin Elmer HTS-7000 Bio Assay reader was used to measure the absorbance of the assays. UV-Vis absorbencies were determined with a Beckman-Coulter DU500 spectrometer.

Aβ fibrils were grown in vitro from synthetic AD-related Aβ peptides (residues 1-42). Fibrils were characterized by atomic force microscopy. Images indicated the presence of fibrils that were consistent with literature reports (Hilbich, C., et al., J. Mol. Biol. 1992, 228: 460.) in terms of size (5-10 nm in diameter and >400 nm long) and in terms of morphology (single fibrils and bundles of fibrils). The wells of commercial 96-well plates were coated with freshly prepared Aβ fibrils and the fibrils were incubated with solutions of ThT. After removal of excess ThT, the ThT-coated fibrils in the wells were treated with a monoclonal anti-Aβ IgG (clone 6E10, derived from residues 3-8 of Aβ peptide as antigens). The interaction of the anti-Aβ IgG with the ThT-coated Aβ fibrils was quantified using an ELISA-based assay.

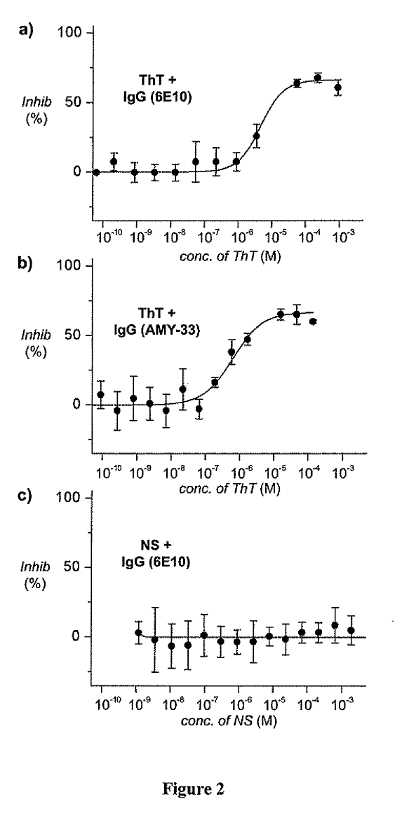

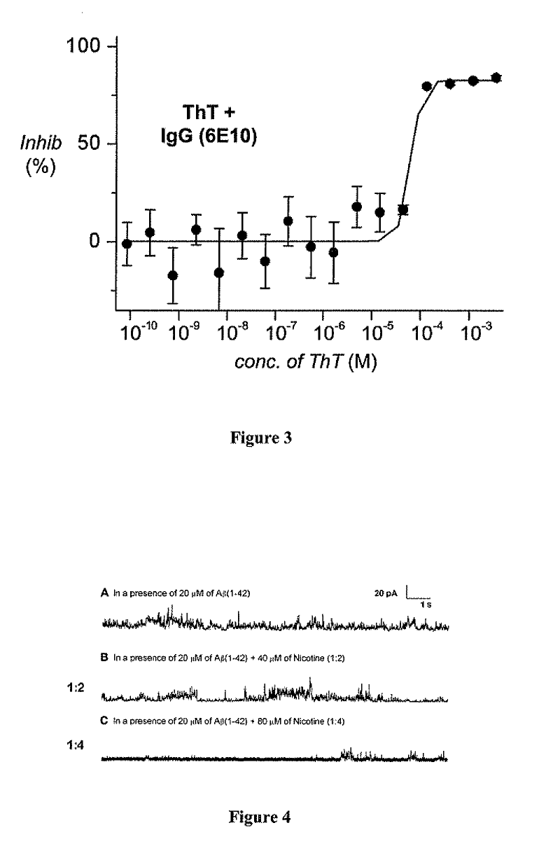

FIG. 2, panel a, shows that ThT had an inhibition concentration corresponding to 50 percent inhibition (IC₅₀) of 5 μM for the binding of the anti-Aβ IgG (clone 6E10, 0.16 μg mL⁻¹) to the Aβ fibrils (deposited from solutions containing 1.3 μM Aβ peptide). Because the final concentration of the Aβ fibrils deposited in the wells was not determined, the Aβ fibrils (1.3 μM) were also incubated with solutions of ThT prior to depositing the coated fibers into the wells and an IC₅₀ of 60 μM was measured (See FIG. 3). For comparison, an IC₅₀ of ˜1 μM was observed when a 0.3 μM solution of Aβ peptide was used in the control procedure. Therefore, it would be expected that observed IC₅₀'s would be different using the two procedures. A total inhibition of 65% of the interaction between this IgG and Aβ fibrils was measured when the fibrils were incubated with a 50 μM solution of ThT. Zero percent inhibition was defined as the UV-Vis signal observed when the assay is run in the absence of ThT and 100 percent inhibition was defined as the UV-Vis signal observed when the assay is run in the absence of both amyloid fibril and ThT. Solutions of ThT with concentrations higher than 50 μM did not increase the total inhibition of the IgG-Aβ fibril interactions above 65%. Coating the fibrils in solutions of ThT (>100 μM of ThT) prior to deposition into wells resulted in a maximum inhibition of ˜80% of the protein-amyloid interactions. Exposing the ThT-coated Aβ fibrils to prolonged washing steps (from 0-4 hrs) with PBS buffer prior to incubation with primary anti-Aβ IgG did not affect the total amount of inhibition of the IgG-amyloid interactions, suggesting the rate of unbinding of ThT from the Aβ fibrils is slow relative to the timescale of the binding assay.

### Example 2

To demonstrate that this surface-coatings approach can extend to other proteins that bind to Aβ fibrils, the ability of ThT to inhibit the interaction of Aβ fibrils with an anti-Aβ IgG raised against a different epitope of Aβ peptide (clone AMY-33, derived from residues 1-28 of Aβ peptide as antigens) was tested. An IC₅₀ of 0.4 μM (FIG. 2, panel b) for ThT with this IgG (clone AMY-33) was measured under the same conditions used to assay the first anti-Aβ IgG (clone 6E10). A maximum inhibition of ˜65% of this IgG (clone MY-33)-Aβ fibril interaction was measured with solutions of ThT having concentrations of 10 μM or higher. No inhibition of the interaction between the IgGs and Aβ fibrils in control experiments using 1-naphthol-4-sulfonate were observed (FIG. 2, panel c). Structurally-similar molecules to 1-naphthol-4-sulfonate do not interact with Aβ fibrils. (Villa, S., et al., Farmaco 2003, 58: 929.) This may suggest that binding of the small molecule to the Aβ fibrils is necessary for the observed inhibition with ThT.

Without limitation as to the possible mechanism of action of inhibition, the observed partial inhibition of the IgG-amyloid interactions by ThT may be, for example, due to ThT not binding (or binding differently) to the terminal ends of the Aβ fibrils (ThT is known to bind only to the fibril form of Aβ peptides (LeVine III, H. Arch. Biochem. Biophys. 1997, 342: 306)). It is possible, therefore, that about 35% of the surface area of each Aβ fibril (presumably localized near the ends of Aβ fibril) may still be accessible for binding by anti-Aβ IgGs even after coating the surface of Aβ fibrils with ThT. Perhaps molecules that more thoroughly coat the surface of Aβ fibrils compared to ThT may show increased inhibition of protein-Aβ fibril interactions.

These examples demonstrate that ThT can inhibit 65±10% of IgG-Aβ fibril interactions. The generation of protein-resistive surface coatings on amyloid fibrils with small molecules may lead to new therapeutic strategies for the inhibition of harmful protein-amyloid interactions in neurodegenerative diseases.

Additional references: Yan, S. D. et al. Nature 1997, 389, 689: Yan, S. D.; Fu, J.; Sot, C.; Chen, X.; Zhu, H.; Al-Mohanna, F.; Collison, K.; Zhu, A.; Stem, E.; Saido, T.; Tohyama, M.; Ogawa, S.; Roher, A.; Stern, D. Nature 1997, 389, 689: Yan, S. D. et al. J. Biol. Chem. 1999, 274, 2145: Yan, S. D.; Shi, Y.; Zhu, A.; Fu, J.; Zhu, H.; Zhu, Y.; Gibson, L; Stern, E.; Collison, K.; Al-Mohanna, F.; Ogawa, S.; Roher, A.; Clarke, S. G.; Stern, D. M. J. Biol. Chem. 1999, 274, 2145. Lustbader, J. W. et al. Science 2004, 304, 448: Lustbader, J. W.; Girilli, M.; Lin, C.; Xu, H. W.; Takuma, K.; Wang, N.; Caspersen, C.; Chen, X.; Pollak, S.; Chaney, M.; Trinchese, F.; Liu, S.; Gunn-Moore, F.; Lue, L. F.; Walker, D. G.; Kuppusamy, P.; Zewier, Z. L.; Aranchio, O.; Stern, D.; Yan, S. S. D.; Wu, H. Science 2004, 304, 448. Yan, S. D. et al. Nature 1996, 382, 685: Yan, S. D.; Chen, X.; Fu, J.; Chen, M.; Zhu, H.; Roher, A.; Slattery, T.; Zhao, L.; Nagashima, M.; Morser, J.; Migheli, A.; Nawroth, P.; Stern, D.; Schmidt, A. M. Nature 1996, 382, 685.

### Example 3

### Assay Methods

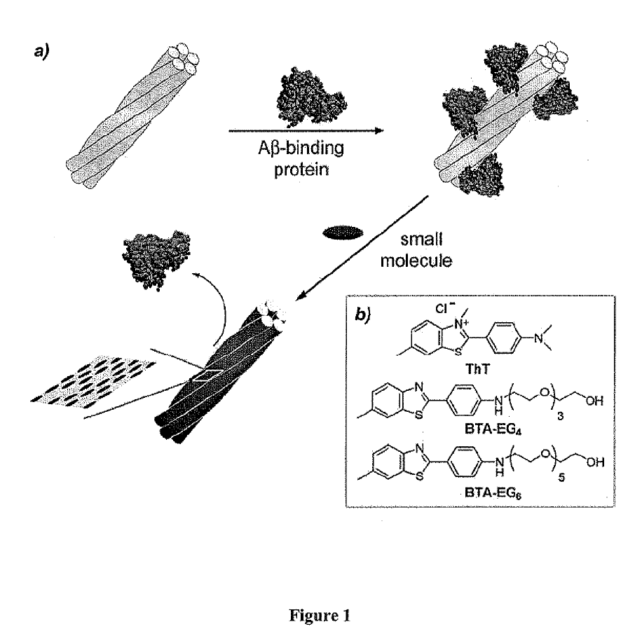

Aβ fibrils were grown from synthetic Aβ(1-42) peptides (Biopeptide Co, LLC, San Diego, Calif., USA) by dissolving 30 μg of peptide in 90 μL of water and incubating at 37° C. for 72 hours. Fibrils were characterized by atomic force microscopy. Images indicated the presence of fibrils (FIG. 1, panel a) that were consistent with literature reports in terms of size (5-10 nm in diameter and a >400 nm long) and in terms of morphology (single fibrils and bundles of fibrils).

The wells of commercial 96-well plates were coated with freshly prepared Aβ fibrils. Each well of a 96 well plate (Titertek®, Huntsville, Ala., USA) was coated for 12 hours with 50 μL of a 5.8 μg/mL (1.3 μM) solution of Aβ peptides (present in fibril form) in phosphate buffered saline (PBS, 10 mM NaH₂PO₄/Na₂HPO₄, 138 mM NaCl, 2.7 mM KCl, pH=7.4). After removal of the excess sample, 50 μL of thioflavin T or 1-naphthol-4-sulfonate solutions in PBS buffer (various concentrations were obtained by diluting a stock solution with PBS buffer) were incubated in the wells for 1.5 or 12 hours, followed by removal of the excess solutions. Alternatively, amyloid fibrils were preincubated for 1.5 hours with ThT at various concentrations by adding amyloid fibrils (having a final concentration of 1.3 μg/mL or 5.8 μg/mL) to the ThT solutions. Wells were coated with ThT-bound fibrils by addition of 50 μL of the preincubated solutions per well and incubation for 1.5 hours. Excess solutions were then discarded. To test if the bound Thioflavin T will be removed during the process of the assay, the sample was subjected to extensive washing steps: 300 μL of PBS was added to the wells and equilibrated for 15 minutes, removed, and then repeated as many as 16 times. The following steps were identical for both methods: All wells were blocked for 30 minutes by adding 300 μL of a 1 (w/v) n solution of bovine serum albumin (BSA, Fraction V, OmniPur) in PBS buffer. Wells were washed once with 300 μL of PBS buffer and incubated for an additional 1 hour with 50 uL of a 0.16 ηg/mL or 0.5 μg/mL of anti-Aβ IgG (dilution 1:6000 in BSA/PBS for clone 6E10 or dilution 1:1000 in 1% BSA/PBS for clone AMY-33, respectively). The wells were washed twice with 300 μL PBS buffer and incubated for 45 minutes with 50 μL of the secondary IgG (1 μg/mL, dilution 1:1000 in 1% BSA/PBS), and washed twice with 300 μL PBS buffer. Bound secondary IgGs were detected by the addition of 50 μL of a p-nitrophenyl phosphate solution (1 mg/mL in 0.1M diethanol amine/0.5 mM magnesium chloride). After the desired intensities were achieved, the enzymatic reaction was quenched after 0.5-2 hours by the addition of 50 μL of a 0.25N sodium hydroxide solution. Absorbance intensities were determined at 405 nm using a UV-Vis spectroscopic plate reader (HTS 7000 Bio Assay Reader, Perkin Elmer, Fremont, Calif., USA). Each run was performed five times and averaged. Graphs were plotted and fitted with the sigmoidal curve fitting option in Origin 6.0 (Microcal Software, Inc., Northhampton, Mass., USA).

Sodium chloride and sodium dihydrogen phosphate hydrate were purchased from Fisher Scientific. Potassium chloride and sodium hydroxide were purchased from Baker. Magnesium chloride was purchased from Sigma. Diethanolamine, p-nitrophenyl phosphate, and 1-naphthol-4-sulfonic acid (sodium salt) were purchased from Fluka. Thioflavin T (ThT) was purchased from MP Biomedica. All reagents were used without further purification. Water (18.2 μΩ/cm) was filtered through a NANOPure Diamond™ (Barnstead) water purification system before use. Metrology Probe™, Tap 300 (Ted Pella, Inc, Redding, Calif., USA) probe tips were used for AFM measurements.

As primary IgGs against Aβ, monoclonal anti-Aβ IgG (clone 6E10, mouse, derived from residues 3-8 of Aβ peptide as antigens,) was obtained from Abcam, Cambridge, Mass., (Lot #79040) and anti-Aβ IgG (clone AMY-33, mouse, derived from residues 1-28 of Aβ peptide as antigens,) was purchased from Zymed Laboratories Inc, South San Francisco, Calif., (Lot #40487378). The secondary anti-mouse IgG (anti-mouse IgG H+L conjugated with alkaline phosphatase, polyclonal, from rabbit) was purchased from Abcam, Cambridge, Mass., (Lot #71496 or #95504). All ELISA based procedures were done at 25° C. unless otherwise stated.

For imaging of Aβ fibrils by atomic force microscopy, 10 μL of an Aβ solution in distilled water (0.33 mg/mL) was placed on freshly cleaved mica (SPI, Westchester, Pa., USA) for 2 minutes. The solution was wicked off with filter paper and the sample was washed twice with 10 μL of water. The sample was then dried under vacuum and imaged using a DI Nanoscope-IV Multimode AFM (Veeco, Santa Barbara, Calif., USA) in tapping mode under ambient conditions.

### Example 4

Screening Compounds for Inhibition of Aβ Fibril: All incubation steps are done at 25° C. unless stated otherwise. Phosphate buffered saline (PBS, 10 mM sodium phosphate, 138 mM sodium chloride, 2.7 mM potassium chloride, pH 7.4) and potassium phosphate buffer (KPi, 200 mM potassium phosphate, 10 mM mercaptoethanol, pH 6.6) are prepared fresh for each experiment.

Growth of Aβ fibrils: Aβ fibrils are grown from synthetic Aβ (1-42) peptides by incubating the peptides (74 μM) in ultrapure water at 37° C. for 72 hours. Fibrils are characterized by electron and scanning probe microscopy. (P. Inbar, J. Yang, Bioorg. Med. Chem. Lett. 2006, 16(4), 1076-1079).

Recombinant expression of ABAD: ABAD is obtained from an external contract laboratory (e.g., Commonwealth Biotechnologies, Inc., Richmond, Va.) that cloned the DNA for ABAD (genebank number AF035555), and expressed and purified the protein from a 1 L culture of E. coli using a literature protocol. (Yan., S. D., et al., J. Biol. Chem. 1999:274:2145-2156); S. D. Yan, Y. Shi, A. Zhu, J. Fu, H. Zhu, Y. Zhu, L. Gibson, E. Stern, K. Collison, F. Al-Mohanna, S. Ogawa, A. Roher, S. G. Clarke, D. M. Stern, J. Biol. Chem. 1999, 274(4), 2145-2156; S. D. Yan, et al., Biochim. Biophys. Acta 2000:1502:145-157). N-terminal sequencing using 15 cycles of Edman degradation is consistent with the predicted sequence of the protein.

Qualitative determination of the binding of Aβ fibrils to catalase and ABAD: The wells of a 96 well plate are coated with catalase or ABAD by incubating each well for 2 hours with 50 μL of a solution of catalase (24 nM in 1% BSA/PBS buffer) or ABAD (1.0 μM, in 1% BSA/KPi buffer). After removal of the solutions containing excess catalase or ABAD, all wells are blocked for 60 minutes using 300 μL of a solution containing 1% BSA in PBS buffer to suppress non-specific adsorption of IgGs to the wells. Wells are washed with 300 μL of PBS buffer and incubated for 2 hours with 50 μL of solutions containing Aβ fibrils (various concentrations are obtained by diluting a stock solution of 49 μM Aβ fibrils) in 1% BSA/PBS buffer. Wells are washed twice with 300 μL of PBS buffer and each well is incubated for an hour with 50 μL of a solution containing a mouse monoclonal anti-Aβ IgG (clone 6E10, lot #145271, 1.1 nM in 1% BSA/PBS).

The amount of bound monoclonal IgGs is quantified by removing the excess solution, washing the wells twice with 300 μL of PBS buffer and by incubating for 45 minutes with 50 μL of a polyclonal secondary rabbit IgG (anti-mouse IgG, 6.8 nM in 1% BSA/PBS) conjugated with alkaline phosphatase, followed by two washes with 300 μL of PBS buffer. The relative amount of secondary IgG bound in each well is quantified by adding 50 βL of a solution containing p-nitrophenyl phosphate (NPP, 2.7 mM, in 0.1 M diethanol amine/0.5 mM magnesium chloride, pH 9.8) to each well. The enzymatic hydrolysis reaction of NPP by alkaline phosphatase is quenched after 45 minutes by adding 50 μL of 0.25 N sodium hydroxide solution to each well and quantifying the concentration of p-nitrophenoxide at 405 nm using a UV-Vis microplate reader. Each data point from this assay represents the average of five independent measurements. Error bars represent standard deviations. Graphs are normalized, plotted and fitted with the sigmoidal curve fitting option in Origin 6.0 (Microcal Software, Inc., Northhampton, Mass., USA).

Inhibition of anti-Aβ IgG-Aβ fibril interactions using small molecules: The wells of a 96 well plate are coated with fibrils formed from Aβ peptides by incubating each well for 2 hours with 50 μL of a 1.3 μM solution of Aβ fibrils in PBS. After removal of solutions containing excess Aβ fibrils, all wells are blocked for 60 minutes using 300 μL of a solution containing 1% BSA in PBS buffer.

The BSA/PBS solutions are discarded and the wells are washed with 300 μL of PBS buffer and incubated with 50 μL of an anti-Aβ IgG (clone 6E10, Lot #145271, 1.1 nM in 1% BSA/PBS) for 1 hour. After removal of solutions containing excess IgG, 50 μL solutions of small molecules in 1% BSA/PBS buffer (for ThT and BTA-EG₆) or 5% DMSO/1% BSA/PBS (for BTA-EG₄) (various concentrations are obtained by diluting a stock solution) are incubated in the wells for 12 hours, followed by removal of solutions containing excess small molecule. The amount of monoclonal IgG present in the wells is quantified as described in the procedure for determining the binding of Aβ fibrils to catalase and ABAD.

Inhibition of catalase-Aβ fibril or ABAD-Aβ fibril interactions using small molecules: The wells of a 96 well plate are coated with fibrils formed from Aβ peptides by incubating each well for 2 hours with 50 μL of a 1.3 μM solution of Aβ fibrils in PBS. After removal of solutions containing excess Aβ fibrils, all wells are blocked for 60 minutes using 300 μL of a solution containing 1% BSA in PBS buffer.

The BSA/PBS solutions are discarded and the wells are washed with 300 μL of PBS buffer and incubated with 50 μL of a human catalase solution (0.20 μM, in 1% BSA/PBS buffer) or 50 μL of an ABAD solution (10 μM, in 1% BSA/KPi) at 37° C. for 3 hours or at 25° C. for 2 hours respectively. After removal of solutions containing excess catalase or ABAD, 50 μL solutions of small molecules in 1% BSA/PBS buffer (for ThT and BTA-EG₆) or 5% DMSO/1% BSA/PBS (for BTA-EG₄) (various concentrations are obtained by diluting a stock solution) are incubated in the wells for 12 hours, followed by removal of solutions containing excess small molecule.

The wells are then washed twice with 300 μL of a solution containing 1% BSA in PBS and each well is incubated for 1 hour with 50 μL of a solution of a monoclonal mouse anti-catalase IgG (clone 1A1, lot #93195, 2.2 nM in 1% BSA/PBS) or 50 μL of a solution of a monoclonal mouse anti-ABAD IgG (clone 5F3, lot #103614, 1.3 nM in 1% BSA/PBS buffer). The amount of monoclonal IgG present in the wells is quantified as described in the procedure for determining the binding of Aβ fibrils to catalase and ABAD.

### Example 5

Synthesis of Nicotine Derivatives: Nicotine, a major component of tobacco, has interesting advantages as a lead structure for development of therapeutics for AD due to: 1) its low molecular weight and low structural complexity; 2) its known blood-brain barrier permeability; 3) its known biocompatibility at low concentrations; and 4) a reported inverse relationship between smoking and AD. (Nyback, H., Halldin, C., Ahlin, A., Curvall, M. & Eriksson, L. Psychopharmacology (Berl) 115, 31-6 (1994); Hukkanen, J., Jacob, P., 3rd & Benowitz, N. L. Pharmacol Rev 57, 79-115 (2005); Graves, A. B. et al. Int J Epidemiol 20 Suppl 2, S48-57 (1991).)

A general schema for the synthesis of nicotine derivatives is set out below:

Example of the Synthesis of a Halogenated Derivative of Nicotine

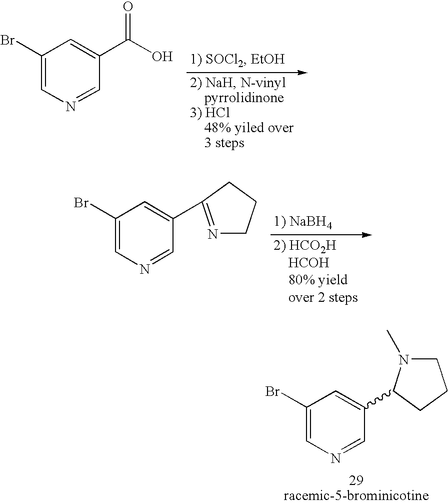

Example of the Synthesis of an Oligoethylene Glycol Derivative of Nicotine

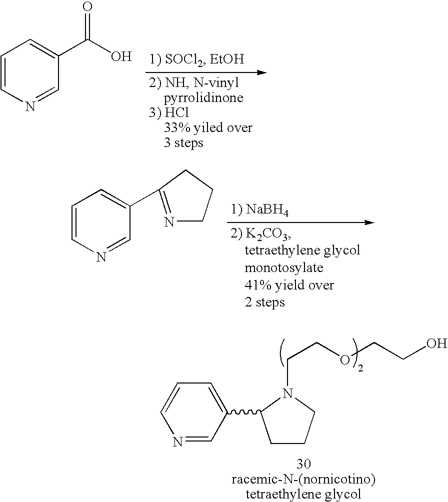

Example of the Synthesis of an Ester Derivative of Nicotinic, Picolinic, or Isonicotinic Acid

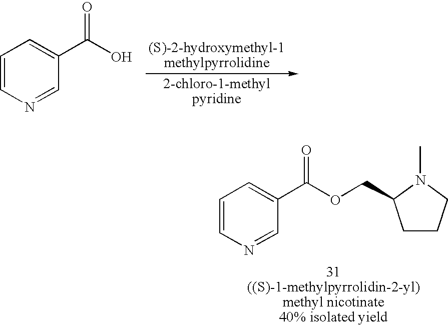

Experimental Details for the Preparation of Nicotine Derivatives: All synthetic reagents were from Aldrich, Fisher Scientific, TCI America, Alfa Aesar or Fluka and were used as received. NMR spectra were obtained on Varian spectrometers (¹H: 300 MHz, 400 MHz). Chemical shifts are reported in ppm relative to residual solvent.

### Ethyl 5-bromonicotinate

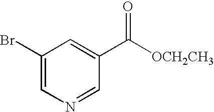

5-Bromonicotinic acid (2.66 g, 13.1 mmol) in excess thionyl chloride (6 mL, 82.3 mmol) was brought to reflux conditions and left to stir overnight. The reaction mixture was cooled to room temperature, placed in an ice bath, and excess ethanol (7 mL) was added to the mixture portion-wise, with stirring. The reaction mixture was returned to reflux conditions and stirred for 2 h. The solution was then cooled to room temperature and solvent removed en vacuo and redissolved in dichloromethane to produce a suspension. 10% NaHCO3 was added with stirring to obtain a pH=8. The dichloromethane layer was removed and the aqueous layer was extracted 2 more times with dichloromethane. The organic fractions were combined, back extracted with water, extracted with brine, and dried over Na2SO4. After filtration, the solvent was removed in vacuo and used without further purification (2.99 g, 99% yield). ¹H NMR (300 MHz, CDCl3) δ ppm 9.04 (d, 1H), 8.75 (d, 1H), 8.34 (dd, 1H), 4.35 (d, 3H) , 1.34 (t, 2H).

### 5-Bromomyosmine

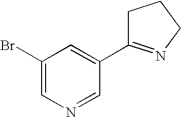

Prepared as in Jacob, Peyton III, J. Org. Chem. 1982, 4165-4167, with slight modifications. To a stirred solution containing ethyl 5-bromonicotinate (3.49 g, 15.2 mmol), N-vinylpyrrolidinone (2.03 g, 18.2 mmol, 1.2 equiv) and dry THF (5.1 mL) under N2, NaH (0.511 g, 21.3 mmol, 1.4 equiv) in 14.2 mL dry THF was added. The reaction was left to stir for 10-15 min until the exothermic reaction was complete and then brought to reflux conditions. After 1 h, the reaction mixture was allowed to cool to room temperature and 4.47 mL of 4.7 M HCl (2 equiv) was added with stirring. The solvent was removed en vacuo and 10.7 mL of 4.7 M HCl (3.33 equiv) was added to the residue. The solution was brought to reflux conditions and left to stir for 18 h. After cooling the reaction mixture to room temperature, 50% NaOH was added portion-wise, with vigorous stirring, to make the solution basic and a thick precipitate formed. The basic solution was extracted twice with dichloromethane. The organic fractions were combined and solvent removed in vacuo. Crude material (1.80 g, 8.0 mmol, 53% crude yield) was used for further reactions. A fraction of the material was purified for characterization (flash chromatography on Si gel, 2% MeOH/CH₂C₁₂, Rf=0.21). ¹H NMR (300 MHz, CDCl3) δ ppm, 8.82 (d, 1H), 8.65 (d, ¹H), 8.29 (t, 1H), 4.04 (tt, 2H), 2.88 (m, 1H), 2.03 (m, 2H). LCMS (ESI+), m/z [M+H]+=225.14.

### 5-Bromonornicotine

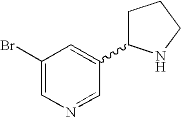

Prepared according to methods known to the skilled artisan with some modifications as described below. A solution containing 5-bromomyosmine (1.8 g, 8.0 mmol) and 20 mL of 4:1 methanol/acetic acid was cooled with a dry ice/acetone bath. Sodium borohydride (0.67 g, 18 mmol, 2.2 equiv) was added to the cooled solution and the solution was left to stir for 1 h in the cold bath. The reaction mixture was then warmed to room temperature over a period of 30 min and continued to stir for 30 min at room temperature until the evolution of H² gas was not observed. The solvent was removed en vacuo and 15 mL of H₂O was added to the residue. The solution was made basic (pH>11) using 50% NaOH with vigorous stirring. A precipitate forms as the solution becomes basic, and the addition of NaOH is arrested once the solution becomes clear. The solution was extracted 3 times with CH₂C₁₂, the organic layers were combined, washed with brine, and dried over K₂CO₃. The solution was filtered and solvent removed in vacuo to resulting in a yellow liquid. The compound was purified by Kugelrohr distillation (145-158° C., 0.7 mmHg) to produce a clear and colorless liquid (1.5 g, 6.5 mmol, 81% yield). ¹H NMR (400 MHz, CDCl3) δ ppm 8.48 (d, 1H), 8.44 (d, 1H), 7.86 (d, 1H), 4.13 (t, 1H), 3.13 (m, 1H), 3.03 (m, 1H), 2.18-1.59 (m, 4H). LCMS (ESI+), m/z [M+H]+=227.00

### 5-Bromonicotine

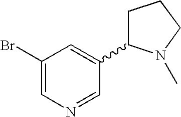

Prepared according to Cosford, N. D. P., et al., J. Med. Chem. 1996, 3235-3237. A mixture of 5-Bromonornicotine (0.169 g, 0.764 mmol) 98% formic acid (4 mL) and 37% aqueous formic acid (2 mL) was heated to 80° C. and left to stir overnight. The reaction mixture was allowed to cool to room temperature and the solvent was reduced to ˜⅓ the original volume. Water and conc. HCl were added to the remaining mixture to obtain a pH of 3. Dichloromethane was added to the mixture with vigorous stirring and the solution was filtered through a pad of celite to get rid of the emulsion. The CH₂C₁₂ layer was removed and the aqueous layer was extracted 2× with CH₂C₁₂. The aqueous layer was made basic to a pH of 12 with 50% NaOH. The basic layer was then extracted 3 times with CH₂C₁₂, all organic fractions were combined, and dried over Na₂SO₄ and filtered. The solvent was removed in vacuo and the target compound was purified by flash chromatography (Si gel, 20:1 CHCl₃/MeOH, Rf=0.31) to obtain # as an oil (0.158 mg, 0.655 mmol, 86% yield). ¹H NMR (400 MHz, CDCl₃) δ ppm 8.49 (d, 1H), 8.38 (d, 1H), 7.81 (t, 1H), 3.17 (dt, 1H), 3.04 (t, 1H), 2.26 (m, 1H) 2.15 (m, 1H), 2.12 (s, 3H), 1.89-1.65 (m, 3H). LCMS (APCI+), I [M+H]+=241.04.

### Myosmine

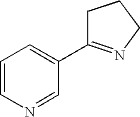

Myosine was prepared following the same procedure for the preparation of 5-Bromomyosmine to produce 5.73 g of crude material (39.2 mmol, 74% crude yield). The compound was used as is without further purification. ¹H NMR (400 MHz, CD₃OD) δ ppm 8.93 (dd, 1H), 8.61 (dd, 1H), 8.21 (ddd, 1H), 7.50 (ddd, 1H), 4.02 (tt, 2H), 3.60 (q, 1H), 3.01 (m, 2H), 2.06 (m, 2H), 1.16 (dd, 2H). LCMS (ESI+) m/z [M+H]+=147.18.

### Nornicotine

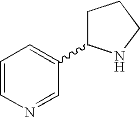

Myosmine was reduced using the same procedure for the preparation of 5-Bromonornicotine. Nornicotine was purified by Kugelrohr distillation (138-150° C., 0.7 Torr) to produce a clear and colorless oil (0.520 g, 3.51 mmol, 72% yield). ¹H NMR (400 MHz, CDC13) δ ppm 8.53 (d, 1H), 8.42 (dd, 1H), 7.65 (m, 1H), 7.21 (dd, 1H), 4.10 (t, 1H), 3.15 (m, 1H) 3.00 (m, 1H), 2.18 (m, 1H) 1.94-1.56 (m, 3H). LCMS (ESI+), m/z [M+H]+=149.08.

### 2-(2-(2-(2-(2-(pyridin-3yl)pyrrolidin-1-yl)ethoxy)ethoxy)ethoxy)-ethanol

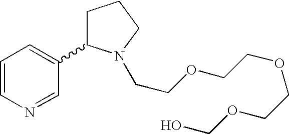

Nornicotine (48.0 mg, 0.320 mmol) tetraethylene glycol mono(p-toluenesulfonate) (Bauer, H., et al., H. A., Eur. J. Org. Chem. 2001, 3255-3278.) (117 mg, 0.336 mmol, 1 equiv) and anhydrous potassium carbonate (186 mg, 1.35 mmol, 4 equiv) in dry acetonitrile was brought to reflux conditions under N₂, and left to stir overnight. Purification by flash chromatography (Si gel, gradient 2-4% NEt3/10% MeOH/CH₂Cl₂, Rf=0.21 at 2% NEt3) produced # as an oil (64 mg, 0.197 mmol, 57% yield). ¹H NMR (400 MHz, CDCl3) δ ppm 8.46 (d, 1H), 8.40 (dd, 1H), 7.72 (dt, 1H), 7.20 (dd, 1H), 3.66 (m, 2H), 3.60-3.51 (m, 8H), 3.47-3.31 (m, 5H), 3.25 (t, 1H), 2.68 (m, 1H), 2.25 (m, 2H), 2.11(m, 1H), 1.96-1.60 (m, 3H). LCMS (ESI+), m/z [M+H]+=325.14.

### (s)-1-Methylpyrrolidine-2-methyl nicotinate

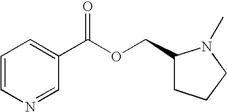

Nicotinic acid (321 mg, 2.60mmol) was added to 12 mL of dry dichloromethane under N₂. The mixture was stirred and (s)(−)1-methyl-2-pyrrolidinemethanol (0.31 mL, 2.61 mmol) was added, immediately followed by triethylamine (0.81 mL, 5.81 mmol). 2-chloro-1-methyl pyridinium iodide (820 mg, 3.21 mmol) was added to the above mixture and the reaction mixture was left to stir overnight. The solvent was removed en vacuo and the crude product was purified by flash chromatography (Si gel, 1% NEt3 in EtOAc, Rf=0.25). The product was further purified by Kugelrohr distillation (60° C., 0.7 Torr). The product was isolated as a highly viscous oil and stored under nitrogen gas at 4° C., (36.3% yield). ¹H NMR (400 MHz, CDCl₃) δ 9.23 (s, 1H), 8.78 (d, 1H), 8.31 (d, 1H), 7.39 (t, 1H), 4.30 (m, 2H), 3.08 (t, 1H) 2.48 (s, 3H), 2.30 (q, 1H) 2.01 (m, 1H), 1.67(m, 4H). LCMS (ESI+), m/z [M+H]+=221.11.

### (s)-1-Methylpyrrolidine-2-methyl picolinate

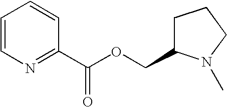

Picolinic acid (322 mg, 2.62 mmol) was added to 14 mL of dry dichloromethane under N₂. The mixture was stirred and (s)(−)1-methyl-2-pyrrolidinemethanol (0.31 mL, 2.61 mmol) was added, immediately followed by triethylamine (0.85 mL, 6.10 mmol). 2-chloro-1-methyl pyridinium iodide (840 mg, 3.29 mmol) was added to the above mixture and left to stir overnight. The solvent was removed en vacuo and the crude product was purified by flash chromatography (Si gel, 1% NEt3 in EtOAc, Rf=0.29) The product was further purified by Kugelrohr distillation (60° C., 0.7 Torr). The product was isolated as viscous oil and stored under nitrogen gas at 4° C. (40.1% yield). ¹H NMR (400 MHz, CDCl₃) δ 8.76 (d, 1H), 8.11 (d, 1H) 7.82 (t, 1H), 7.46 (t, 1H), 4.34 (m, 2H), 3.08 (t, 1H), 2.48 (s, 3H), 2.26 (m, 2H), 1.67 (m, 4H). LCMS (ESI+), m/z [M+H]+=220.97

### Example 6

Inhibition of Aβ Fibril Protein Binding by Nicotine Derivatives: Nicotine derivatives were assayed for their ability to inhibit Aβ fibril protein binding essentially as discussed in Example 4, and throughout the present application, using anti-Aβ IgG.

Synthesis of nicotine derivatives: 7-10 (Scheme) esters 13-15 (Scheme):

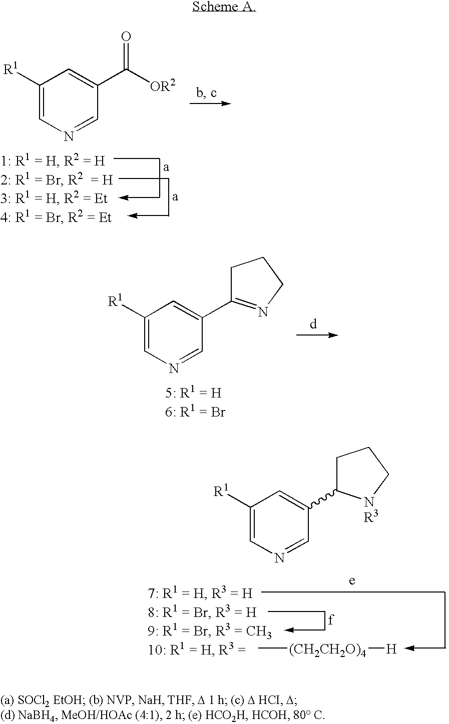

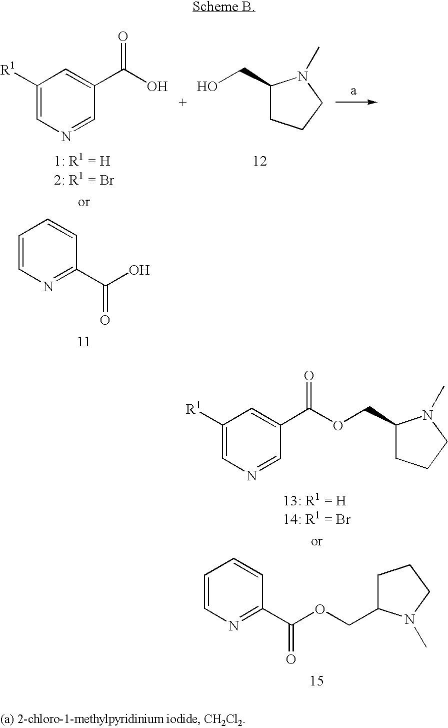

FIG. 8 and Table 3 below present the results of these inhibition assays:

| | % Max | Conc at Max | |
|---|---|---|---|
| Compound | Inhib. | Inhib., mM | IC₅₀, mM |
| (−)-Nicotine | 84 | 1,000 | 24 |
| 7 | 98 | 700 | 80 |
| 8 | 83 | 700 | 33 |
| 9 | 70 | 450 | 19 |
| 10 | 81 | 100 | 4 |
| 13 | 54 | 50 | 11 |
| 14 | 68 | 50 | 3 |
| 15 | 51 | 50 | 5 |

Table 4 below presents an analysis of the potential of the nicotine derivatives to passively diffuse through cell membranes and through the blood-brain barrier.

| | MW | | | | | |
|---|---|---|---|---|---|---|
| | [g/ | LogP | | No. of H-bond | | |
| Compound | mol] | expt'l | calc'd | HA | HD | TPSA |
| ideal | <500 | 1-3 (ideal ~2) | | <10 | <5 | <120 |
| nicotine | 162 | 0.20 ± 0.01 | 1.09 | 2 | 0 | 16.1 |
| 7 | 148 | −0.93 ± 0.04 | 0.85 | 2 | 1 | 24.9 |
| 8 | 241 | 2.1 ± 0.3 | 2.05 | 2 | 0 | 16.1 |
| 9 | 227 | 0.31 ± 0.02 | 1.80 | 2 | 1 | 24.9 |
| 10 | 324 | −0.41 ± 0.05 | −0.059 | 6 | 1 | 64.1 |
| 13 | 220 | −0.50 ± 0.02 | 0.95 | 4 | 0 | 42.4 |
| 14 | 299 | | 1.91 | 4 | 0 | 42.4 |
| 15 | 220 | 0.69 ± 0.08 | 1.02 | 4 | 0 | 42.4 |

### Example 7

Functional Ion Channel Inhibition Assays: A functional assay has been developed to investigate the inhibition of neurotoxic ion channel activity of Aβ peptides in reconstituted membrane bilayers and in aneuronal cell line by small molecules. The assay is based on ultra-sensitive electrophysiological recordings of the ion channel activity of Aβ. This ion channel-activity assay is designed to determine whether small molecules that bind to Aβ fibrils will inhibit ion channel activity. Compounds that inhibit ion channel activity are likely candidates for therapeutics and diagnostic agents for amyloid-associated diseases. This Example presents results that nicotine, tannic acid, and Congo Red are able to inhibit the ion channel activity of Aβ oligomers in planar lipid bilayers.

Ion Channel Assay—Lipid Bilayer:

1. The lipid mixture was made from POPE:POPG (Avanti Polar Lipids) at 25 mg/ml (1:1) in Heptane. The pretreatment lipid solution was POPE:POPG 20 mg/mL in Hexane.

2. The bilayer was formed in classic bilayer cups and chamber (Warner Instruments). This 2-part system consists of a black Delrin chamber and a cup of Delrin. Cups and chambers are designed such that addition of equal volumes to the cup and chamber (cis and trans sides) results in a balanced solution height, minimizing any pressure gradients across the bilayer membrane.

3. The bilayer was formed over a 250 μm hole in a partition separating two Delrin compartments, the so called “painting technique.” First, droplets of pretreatment lipid solution were placed on both sides of the hole, using 100 uL-Hamilton syringe. Once the droplet of hexane evaporates, both chambers (cis and trans) were symmetrically filled with 900 uL of buffer solution, 100 mM KH2PO4/K2HPO4 pH 7.4, at room temperature.

4. When the bilayer set-up was already in place with Ag/AgCl electrodes in Faraday cage (a bilayer workstation which provides critical shielding of electromagnetic interference from outside sources and isolate vibration), the lipid solution (POPE:POPG in Heptane) was painted directly over the hole by using a thin paintbrush. A researcher gently blew underneath the hole by using the Pasteur pipette to thin out the bilayer until the appropriate capacitance was obtained (80-120 pF).

5. A voltage of ±100 mV was applied for at least 10 minutes to test stability of lipid bilayer.

Amyloid Beta Protein:

Aβ(1-42) (Biopeptides) was initially dissolved in deionized water at 1 mg/ml (221.5 μM), and stored at −20° C. The stock solution is aliquotted to sufficient amount for each time use (90 uL). After the stable bilayer is constituted, the Aβ(1-42) solution was added to the trans side of the chamber to obtain a final concentration of 37 μM. The solution was mixed well in the chamber under stirring for 5 minutes

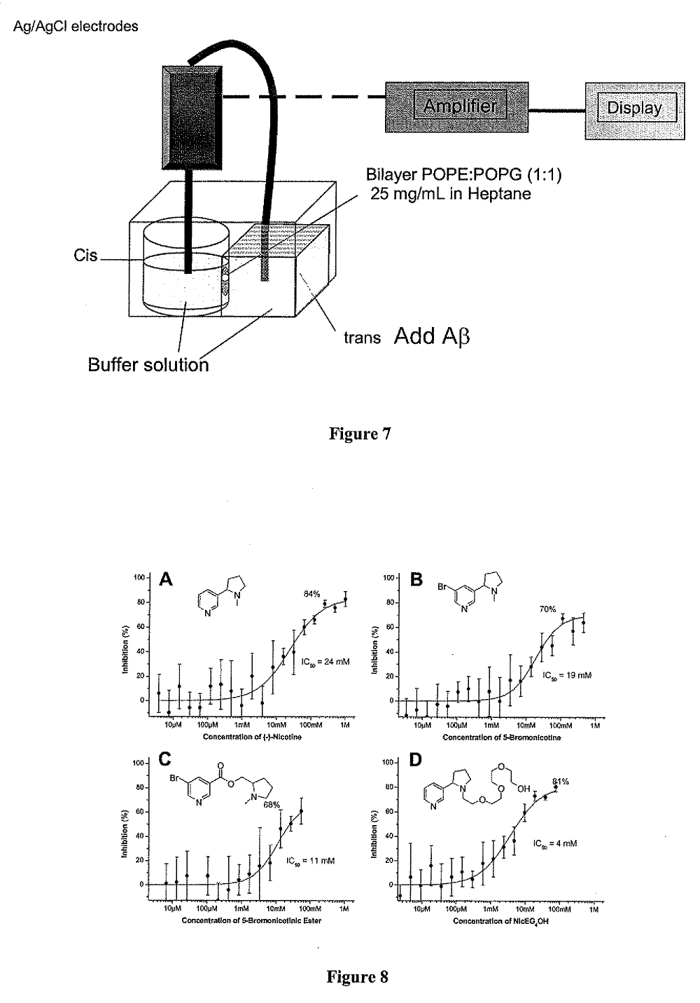

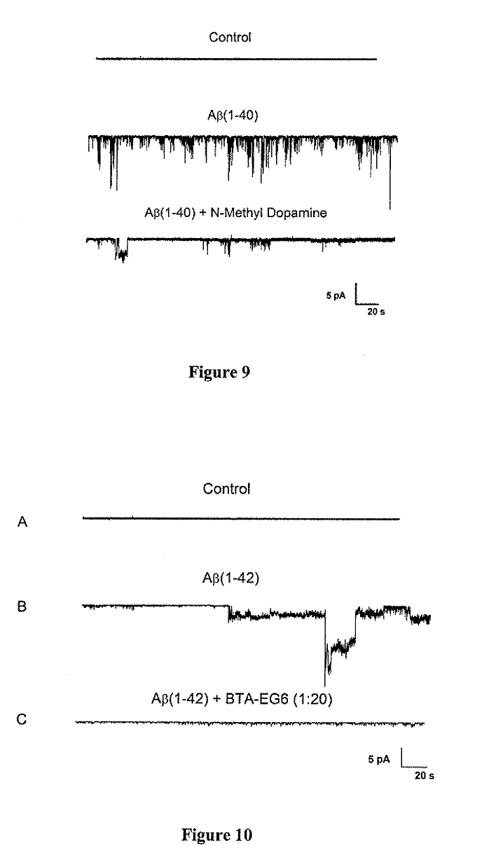

As shown in FIG. 7, the cis side of the chamber was directly connected to the headstage, while the trans side of the chamber was electrode-grounded to Ag/AgCl electrodes.

Inhibition Agents:

Congo Red (Sigma) was dissolved in DI Water to achieve a concentration of 2.5 mg/ml (3.58 mM) as a stock solution. (−) Nicotine Hemasulfate (Sigma) was diluted to 1.89 mM in DI Water. Tannic Acid (Riedel-de Haen) was diluted to 20 mM in DI Water.

The solution of inhibitory molecule was added to cis and trans sides to make a desired final concentration (1:1 molar ratio) at the same time as Aβ.

For Nicotine, the solution was added after observing current activities of Aβ to obtain 1:1, 1:2, 1:3, 1:4 molar ratio.

FIG. 4 shows that the addition of nicotine resulted in concentration-dependent disruption of Aβ ion channel activity. A molar ratio of nicotine to Aβ peptides of 4:1 disrupted preformed Aβ channels almost completely. Control experiments with molecules known not to bind to Aβ did not result in inhibition of Aβ ion channel activity.

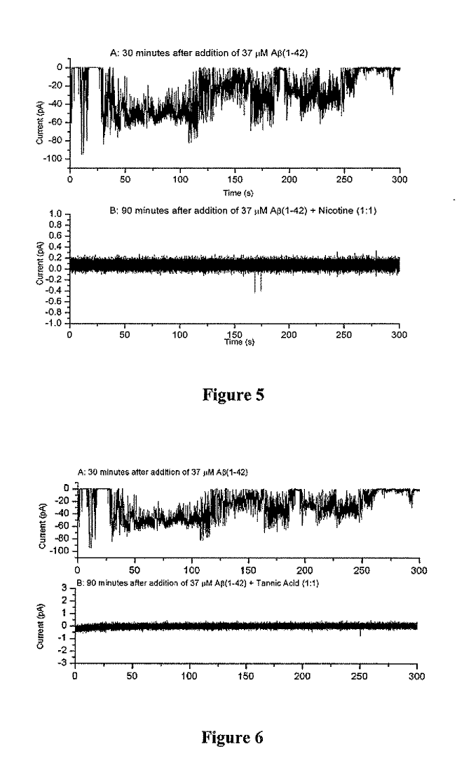

It was observed that the disruption of preformed Aβ ion channels required more nicotine than the inhibition of de-novo formed ion channels. FIG. 5 shows that amolar ration of 1:1 was sufficient to inhibit the de-novo formation of Aβ ion channels. Again, the presence of control molecules had no inhibitory effect on ion channel formation of Aβ.

The experiment shown in FIG. 5 was repeated with Congo Red and tannic acid. Both molecules had been found to bind strongly to aggregated Aβ fibrils. FIG. 6 shows the results with tannic acid, which inhibited ion channel activity of Aβ. Similar results were obtained with Congo Red.

By repeating the experiments with nicotine, tannic acid, and Congo Red several times, it was found that nicotine had the strongest inhibitory effect on Aβ ion channel activity, followed by tannic acid and then by Congo Red.

### Example 8

Quantified Ion Channel Inhibition Assays: A time-averaging method is used to quantify the ion channel current from ion channel-forming antibiotic peptides in planar lipid bilayers. (Blake, S., Mayer, T., Mayer, M. & Yang, J. Chem Bio Chem 7, 433-435 (2006); Mayer, M., Gitlin, I., Semetey, V., Yang, J. & Whitesides, G. M. in preparation (advanced draft) (2006)) This approach is adapted to the analysis of Aβ ion channel activity. Aβ peptides with high purity are obtained (Bachem). Solubilizing agents (e.g. DMSO, TFE, or TFA) (Walsh, D. M., Hartley, D. M., Condron, M. M., Selkoe, D. J. & Teplow, D. B. Biochem J 355, 869-77 (2001)) are then used to ensure that all Aβ peptides are present as monomers and not in a pre-aggregated state of oligomers or fibrils. A first set of experiments may be carried out with Aβ(1-42) as this peptide is important for the neurotoxic mechanism of the disease and is known to form significant ion channel activity in planar lipid bilayers as well as in the membrane of living cells; Aβ(1-40) shares these characteristics but it aggregates more slowly into fibrils and it takes longer before ion channel activity is observed. The ion channel activity of Aβ is quantified by measuring the total transported charge in a given time interval (e.g. 1 min). This experiment is performed multiple time, for example, at least four times, to obtain a reliable average and then repeated at increasing concentrations of the Aβ peptide. As with previous quantitative work on self-aggregating ion channel-forming peptides, it is expected that this approach will yield reliable statistics to establish the total transported charge through Aβ ion channels as a function of the concentration of Aβ. (Blake, S., Mayer, T., Mayer, M. & Yang, J. Chem Bio Chem 7, 433-435 (2006); Mayer, M., Gitlin, I., Semetey, V., Yang, J. & Whitesides, G. M. in preparation (advanced draft) (2006); Mayer, M., Yang, J., Gitlin, I., Gracias, D. H. & Whitesides, G. M. Proteomics 4, 2366-2376 (2004).) The possible dependence of the ion channel activity on the time-lapse after the addition of the Aβ peptides is analyzed. A well-established time-dependence will make it possible to compare the ion channel activity before and after addition of the small molecules with binding affinity for Aβ fibrils. The results obtained with Aβ(1-42) may be compared with the ones obtained with Aβ(1-40). All recordings are carried out at 37° C. using microfabricated planar lipid bilayer setups, which afford reliable and stable low-noise recording conditions. These setups have been developed and optimized. (Blake, S., Mayer, T., Mayer, M. & Yang, J. Chem Bio Chem 7, 433-435 (2006); Mayer, M., Gitlin, I., Semetey, V., Yang, J. & Whitesides, G. M. in preparation (advanced draft) 2006); Mayer, M., Schmidt, C., Giovangrandi, L. & Vogel, H. Eur. Biophys. J. Biophys. Lett. 29, 378 (2000); Terrettaz, S., Mayer, M. & Vogel, H. Langmuir 19, 5567-5569 (2003); Mayer, M. in Physical Chemistry 25-61 (Swiss Federal Institute of Technology, Lausanne, Switzerland, 2000); Schmidt, C., Mayer, M. & Vogel, H. Angew. Chemie Int. Ed. 39, 3137-3140 (2000); Mayer, M., Terrettaz, S., Giovangrandi, L. & Vogel, H. in Biosensors: A practical approach (eds. Cooper, J. M. & Cass, A. E. G.) 153-184 (Oxford University Press, Oxford, 2003).; Mayer, M., Kriebel, J. K., Tosteson, M. T. & Whitesides, G. M. Biophys J 85, 2684-95 (2003).)

Using this quantitative ion channel assay, the ion channel activity of Aβ before and after addition of the Aβ-binding molecules is compared. Dose-response curves of inhibition of ion channel activity as a function of increasing concentrations of small molecule are constructed.

The inhibitory effect of molecules that interfere with the assembly process of peptides to ion channels has been found to typically follow a power law with respect to the concentration of the inhibitory molecule. The inhibitory effect thus is expected to increase strongly (non-linearly) with concentration.

### Example 9

Inhibition of Ion Channel Activity of Aβ in a Neuronal Cell Line: Compounds may be tested for ion channel activity inhibition on the neuronal cell line SH-SY5Y (human neuroblastoma cells). Well-defined concentrations of Aβ are added to the growth media of the cells and the cells are grown for several days. The cytotoxicity of Aβ is measured, for example, at least twice per day by performing MTT assay. (Bollimuntha, S., Ebadi, M. & Singh, B. B. Brain Res 1099, 141-9 (2006); Copeland, R. L., Jr., Leggett, Y. A., Kanaan, Y. M., Taylor, R. E. & Tizabi, Y. Neurotox Res 8, 289-93 (2005); Locke, C. et al. J Neural Transm 105, 1005-15 (1998); Zhao, F. L., Hu, J. H. & Zhu, X. Z. Biol Pharm Bull 29, 1372-7 (2006).) In parallel, cells are incubated with the same concentrations of Aβ but in the presence of small molecules that showed inhibitory effects on Aβ ion channels. Comparison between the two populations establishes the effectiveness of the small molecules to protect SH-SY5Y cells from the cytotoxic activity of Aβ. Two more controls may be run in parallel: in one, only growth medium is added to the cells, and in the other, the small molecules are added to the growth medium. The comparison of the cell viability between those two populations establishes the possible inherent cytotoxicity of the small molecules.

Compounds are also tested on SH-SY5Y cells using patch clamp technology. In these experiments, Aβ and small molecules are added to quantify functionally the inhibitory effect of the small molecules on the ion channel activity in live cells.

### Example 10

Diffusion Across Cell Membranes and the Blood Brain Barrier: In order to assess the potential utility of a compound of the present invention as probes to study protein-amyloid interactions in cellular assays, for use in diagnostic imaging, or for use as therapeutics to treat amyloid-associated diseases, the likeliness of the compounds to passively diffuse across cell membranes and the Blood-Brain-Barrier (BBB) may be estimated. (C. A. Lipinski, F. Lombardo, B. W. Dominy, P. J. Feeney, Adv. Drug Delivery Rev. 1997, 23(1-3), 3-25)

15 μM solutions of small molecule are prepared in 5 mL PBS buffer. 5 mL of octanol is added to each aqueous solution of small molecule and the biphasic layers are mixed by rapid vortexing. The mixture is then centrifuged at 250×g to facilitate the formation of two clear layers. The layers are separated and the absolute concentrations of small molecules in each layer are quantified by measuring the absorbance of the layers at the appropriate nm for each compound. The molar extinction coefficient of the compound in octanol and PBS buffer is determined by comparison to standard calibration curves of known quantities of small molecules dissolved in octanol and PBS buffer. The partition coefficient is expressed as logarithm of the ratio of the concentration in octanol divided by the concentration in PBS (i.e., log P).

Topological polar surface areas are estimated using Molinspiration Cheminformatics software. This web-based software is available on the WorldWideWeb at molinspiration.com/cgi-bin/properties.

The measured log P_{octanol/water} (Klunk, W. E., et al., Life Sci. 2001, 69:1471-1484) and calculated polar surface areas (a) D. E. Clark, J. Pharm. Sci. 1999, 88(8), 815-821; b) J. Kelder, P. D. J. Grootenhuis, D. M. Bayada, L. P. C. Delbressine, J.-P. Ploemen, Pharm. Res. 1999, 16(10), 1514-1519; P. Ertl, B. Rohde, P. Selzer, J. Med. Chem. 2000, 43(20), 3714-3717) may be used to predict the compound's biocompatibility for use in cellular or in in vivo studies.

In other examples of blood brain barrier penetration studies, rabbits are injected with the test compound and the amount in the blood serum and the cerebralspinal fluid is determined after 2,3,6 and 12 hours. Samples are taken under anesthesia as in, for example, Chan, K., et al., Asia Pacific J. Pharm. 1986, 1(1), 41-45.

### Example 11

General Procedure for detecting the binding of small molecules to amyloid fibrils: All incubation steps are done at 25° C. unless stated otherwise. Phosphate buffered saline (PBS, 10 mM sodium phosphate, 138 mM sodium chloride, 2.7 mM potassium chloride, pH 7.4) are prepared fresh for each experiment.

Wells of a 96 well plate are coated with fibrils formed from various amyloid forming peptides (such as α-synuclein, huntingtin, amylin) by incubating each well for 2 hours with 50 μL of a solution of amyloid fibrils in PBS (concentration 0.05-5 μM). After removal of solutions containing excess fibrils, all wells are blocked for 30 minutes using 300 μL of a solution containing 1% BSA in PBS buffer.

The BSA/PBS solutions are discarded and the wells are washed with 300 μL of PBS buffer and 50 μL solutions of small molecules in 1% BSA/PBS buffer (various concentrations can be obtained by diluting a stock solution) are incubated in the wells for 12 hours, followed by removal of solutions containing excess small molecule. The wells are washed twice with 300 μL PBS buffer and incubated with 50 μL of a mouse monoclonal anti-amyloid IgG (IgGs are commercially available from e.g., Abcam, Inc, Cambridge, Mass.) and are raised against the fibril deposited into the wells. Concentrations are optimized and might range from 0.05-10 nM in 1% BSA/PBS) for 1 hour. After removal of solutions containing excess IgG, the relative amount of secondary IgG bound in each well is quantified by adding 50 μL of a solution containing p-nitrophenyl phosphate (NPP, 2.7 mM, in 0.1 M diethanol amine/0.5 mM magnesium chloride, pH 9.8) to each well. The enzymatic hydrolysis reaction of NPP by alkaline phosphatase is quenched after 45 minutes by adding 50 μL of 0.25 N sodium hydroxide solution to each well and quantifying the concentration of p-nitrophenoxide at 405 nm using a UV-Vis microplate reader. Each data point from this assay will represent the average of five independent measurements. Error bars will represent standard deviations. Graphs can be normalized, plotted and fitted with the sigmoidal curve fitting option in Origin 6.0 (Microcal Software, Inc., Northhampton, Mass., USA).

### Example 12

Cytoprotection: Table 5 below provides a summary of cytoprotection and inhibition of Aβ activity.

| Molecule | | Method | Inhibition | |
|---|---|---|---|---|
| 1. | (−) Nicotine | 1 | 1. | (incubated at 2:1), unstable membrane |
| | | | 2. | (incubated at 4:1), membrane lasts ~70 minutes |
| | | | | (average of 4 experiment) |
| | | | Observe no ion channel activity in 4 out of 4 | |
| 2. | Dopamine HCl | 1 | 1. | (incubated at 4:1), membrane lasts ~70 minutes |
| | | | | (average of 4 experiments) |
| | | | Observe no ion channel activity in 3 out of 4 | |
| 3. | Tannic Acid | 1 | 1. | (incubated at 4:1), membrane lasts ~75 minutes |
| | | | | (average of 5 experiments) |
| | | | Observe no ion channel activity in 3 out of 5 | |
| 4. | Curcumin | 1 | 1. | (incubated at 2:1), membrane lasts ~50 minutes |
| | | | | (average of 6 experiments) |
| | | | Observe no ion channel activity in 3 out of 6 | |
| 5. | Salicylic Acid | 1 | 1. | (incubated at 4:1), membrane lasts ~45 minutes |
| | sodium salt | | | (average of 3 experiments) |
| | | | Observe no ion channel activity in 3 out of 3 | |
| 6. | L-(−)- | 2 | 1. | Observe no ion channel activity at 50 μM small |
| | norepinephrine | | | molecule |
| | (+)-bitartrate salt | | | |
| | monohydrate | | | |
| 7. | L-DOPA | 2 | 1. | Observe no ion channel activity at 50 μM small |
| | | | | molecule, membrane broke |
| 8. | N-methyl | 2 | 1. | Observe no ion channel activity at 450 μM small |
| | dopamine | | | molecule |
| | hydrochloride | | | |
| 9. | BTA-EG₄ | 3 | 1. | Observe no ion channel activity at 200 μM small |
| | | | | molecule |
| 10. | BTA-EG₆ | 3 | 1. | Observe no ion channel activity at 200 μM small |
| | | | | molecule |

The following chemical structures correspond to molecules 1-10 as set forth in Table 5 above:

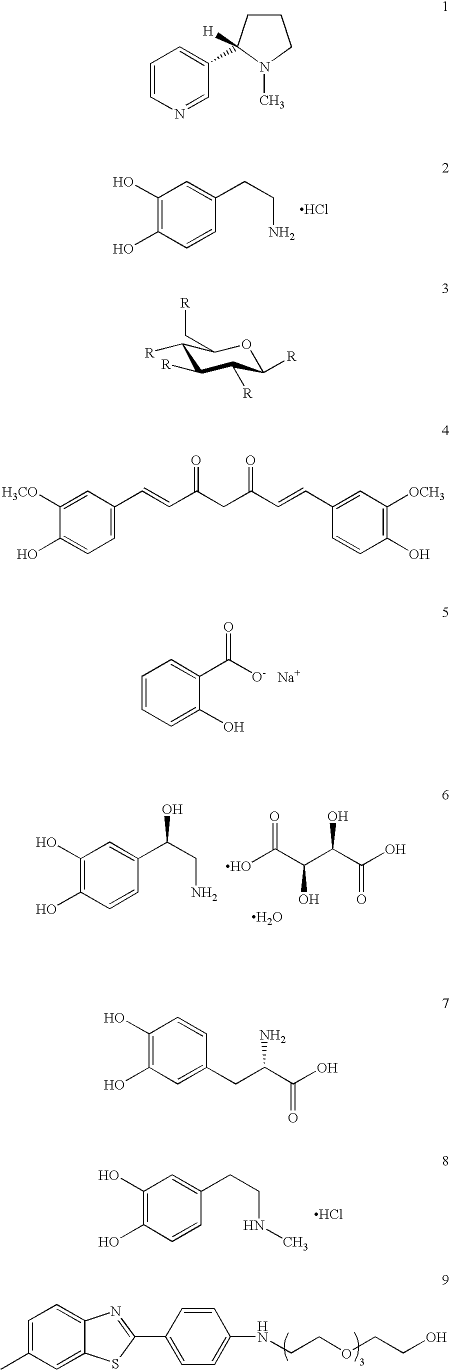

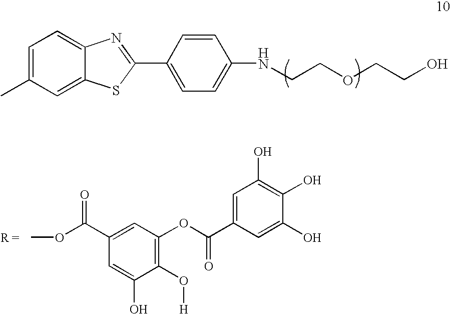

Methods 1, 2, and 3 referred to in Table 5 are discussed in detail below.

Method 1: Aβ(1-42) was initially dissolved in DMSO at 550 μM and diluted to 37 μM final concentration in recording buffer (100 mM K₂HPO₄/KH₂PO₄ pH 7.4). The Aβ sample was pre-incubated at RT for 12-18 hours. The molecules of interest can be pre-incubated with Aβ sample at various concentrations.

Bilayer set-up and recording system: Planar lipid bilayer was formed by the so-called “painting technique” over a 250-μm aperture on a Delrin cup (Warner Instruments) separating two compartments (cis and trans) of a bilayer setup. A lipid mixture of 1-palmitoyl-2-oleoyl phosphatidyletanolamine (POPE): palmitoyl-oleyl-phosphatidylglycerol (POPG) (Avanti Polar Lipids), 1:1 at 25 mg mL-1 in heptane was applied to the aperture which is pre-treated with 2 μL of the same lipid mixture (20 mg mL⁻¹) in hexane on each side. The recording buffer in cis compartment is 100 mM K₂HPO₄/KH₂PO₄ pH 7.4, while in the trans compartment a buffer with pre-incubated Aβ overnight was used.

Method 2: Planar lipid bilayer was formed by the so-called “painting technique” over a 250-μm aperture on a Delrin cup (Warner Instruments) separating two compartments (cis and trans) of a bilayer setup. A lipid mixture of 1-palmitoyl-2-oleoyl phosphatidyletanolamine (POPE) (Avanti Polar Lipids) and Dioleoylphosphatidylserine (DOPS) (Avanti Polar Lipids), 1:1 at 10 mg mL-1 in heptane was applied on the aperture which is pre-treated with 2 μL of the same lipid mixture in hexane on each side. Both compartments were filled symmetrically with 800 μL of 70 mM KCl, 10 mM. A glass pipette with a smooth bent tip was used to blow air bubble under the aperture to thin out the droplet of lipid to obtain a planar lipid bilayer (with capacitance >80 pF). Membrane stability was determined by applying ±100 mV for 10 minutes and monitoring a constant current baseline without instabilities in current. The capacitance of the membrane was monitored throughout the experiment.

Monomerization of Aβ: Aβ powder (Biopeptide, Inc.) was initially solubilized in Hexafluoroisopropanol (HFIP) at 1 mM for 21 hours in a glass vial, with 3 times of vortexing throughout the incubation period. The solution was diluted with cold nanopure water (2:1 H2O:HFIP) vortexed, fractionated in desire amounts, and immediately frozon in a CO₂/acetone bath. Each fraction was covered with parafilm that was punctured twice to allow solvent vapors to escape. The fractions were lyophilized for 2 days to obtain monomerized Aβ.

Preparation of Aβ proteoliposomes: Aβ(1-40) (Biopeptide, Inc.) powder was initially solubilized lyophilized in DiH₂O at 1 mg/mL and stored in −80° C. before use. 20 μL of DOPS was evaporated in CHCl₃ (10 mg mL⁻¹) under vacuum and formed liposomes using hydration method. After obtaining a thin film of lipid, 30 μL of 1M Potassium Aspartate pH 7.2 was added, followed by 5 minutes of bath sonication. The liposome suspension was then mixed with 20 μL of Aβ (1-40) solution (1 mg/mL) and sonicated for 5 minutes.

In order to promote fusion of Aβ proteoliposomes into the bilayer, an ionic gradient of 370 mM KCl was used on the cis side (the side of proteoliposome addition) and 70 mM KCl on the trans side of the bilayer setup. 10-20 μL of the Aβ proteoliposome solution was added to the cis compartment and stirred vigorously for 5-10 minutes. The molecules of interest can be tested by addition to the chamber after observing events.

Method 3: Planar lipid bilayer was formed by the so-called “painting technique” over a 250-μm aperture on a Delrin cup (Warner Instruments) separating two compartments (cis and trans) of a bilayer setup. A lipid mixture of 1-palmitoyl-2-oleoyl phosphatidyletanolamine (POPE) (Avanti Polar Lipids) and 1-palmitoyl-2-oleoyl phosphatidylcholine (POPC) (Avanti Polar Lipids), 1:1 at 10 mg mL⁻¹ in heptane was applied on the aperture which is pre-treated with 2 μL of the same lipid mixture in hexane on each side. Both compartments were filled symmetrically with 800 μL of 70 mM KCl, 10 mM Hepes, pH 7.4. A glass pipette was used with a smooth bent tip to blow air bubble under the aperture to thin out the droplet of lipid to obtain a planar lipid bilayer (with capacitance >80 pF). Membrane stability was determined by applying ±100 mV for 10 minutes and monitoring a constant current baseline without instabilities in current. The capacitance of the membrane was monitored throughout the experiment.

Preparation of Aβ oligomers: Aβ(1-42) (Biopeptide, Inc.) solubilized 1 mg of in 400 μL hexafluoroisopropanol (HFIP) for 15 minutes at room temperature. 100 μL of completely dissolved Aβ(1-42) solution was added in 900 μL of DiH₂O in a siliconized Eppendorf tube and incubated for 15 minutes. The sample was centrifuged at 14,000 g, RT for 15 minutes. After the centrifugation, 950 μL of Aβ solution was transferred to the new siliconized tube, which was cut at the bottom and inverted. The sample was stirred at 500 RPM using a Teflon-coated micro stir bar for 48 hours to remove HFIP, and allows aggregation of Aβ. The molecules can be either added after observing the Aβ activity or pre-incubated with Aβ sample at various concentrations, usually up to 20 fold to Aβ concentration. The new concentration of Aβ(1-42) and/or small molecule was calculated from the remaining volume of Aβ solution after incubation was complete.

The details of one or more embodiments of the disclosure are set forth in the accompanying drawings and the description below. Other features, objects, and advantages will be apparent from the description and drawings, and from the claims.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Singular forms “a”, “an”, and “the” include plural reference unless the context clearly dictates otherwise. Thus, for example, reference to “a subset” includes a plurality of such subsets, reference to “a nucleic acid” includes one or more nucleic acids and equivalents thereof known to those skilled in the art, and so forth. The term “or” is not meant to be exclusive to one or the terms it designates. For example, as it is used in a phrase of the structure “A or B” may denote A alone, B alone, or both A and B.

Unless defined otherwise, all technical and scientific terms used herein have the same meanings as commonly understood by one of ordinary skill in the art to which this invention belongs. Although any methods and systems similar or equivalent to those described herein can be used in the practice or testing of the present invention, the methods, devices, and materials are now described. All publications mentioned herein are incorporated herein by reference for the purpose of describing and disclosing the processes, systems, and methodologies that are reported in the publications which might be used in connection with the invention. Nothing herein is to be construed as an admission that the invention is not entitled to antedate such disclosure by virtue of prior invention.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A method of inhibiting or disrupting Aβ fibril interaction with cellular proteins comprising contacting the Aβ fibril with a compound selected from the group consisting of tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, and BTA-EG₆.

2. The method of claim 1, wherein the compound is selected from the group consisting of nornicotine, 5-bromonornicotine, 5-bromonicotine, and 5-iodonicotine.

3. The method of claim 1, wherein the compound is selected from the group consisting of a nicotinic ester, a 5-bromopicolinic ester, and a picolinic ester.

4. The method of claim 1, wherein the cellular protein is expressed in neural tissue.

5. The method of claim 1, wherein the Aβ fibril interaction with cellular proteins is associated with a neuronal disease.

6. The method of claim 5, wherein the neuronal disease is selected from the group consisting of Alzheimer's disease, Parkinson's disease, Huntington's disease, Down's Syndrome, cerebrovascular amyloidosis, Lewy body dementia, and spongiform encephalopathy.

7. A method of inhibiting or disrupting ion channel activity of beta amyloids associated with a neuronal disease, comprising contacting a beta amyloid with a compound selected from the group consisting of tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, and BTA-EG₆.

8. The method of claim 7, wherein the compound is selected from the group consisting of nornicotine, 5-bromonornicotine, 5-bromonicotine, and 5-iodonicotine.

9. The method of claim 8, wherein the compound is selected from the group consisting of a nicotinic ester, a 5-bromopicolinic ester, and a picolinic ester.

10. A method of preventing or alleviating the symptoms of an amyloid-associated neuronal disease comprising contacting a subject with a compound selected from the group consisting of tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, and BTA-EG₆.

11. The method of claim 10, wherein the compound inhibits or disrupts Aβ fibril interactions with cellular proteins.

12. The method of claim 10, wherein the neuronal disease is selected from the group consisting of Alzheimer's disease, Parkinson's disease, Huntington's disease, Down's Syndrome, cerebrovascular amyloidosis, Lewy body dementia, and spongiform encephalopathy.

13. A method for diagnosing an amyloid associated disease in an individual, comprising administering an Aβ fibril-binding compound to an individual and detecting the binding of the compound to amyloid deposits in the individual, wherein the compound is selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or any combination thereof.

14. A method for detecting amyloid deposits in a subject, comprising administering a pharmaceutical composition comprising a pharmaceutically acceptable carrier and a compound selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or any combination thereof; and detecting the binding of the compound to an amyloid deposit in the subject.

15. The method of claim 14, wherein the amyloid deposit is present in the brain of the subject.

16. A method of preventing or alleviating the symptoms of an amyloid associated disease comprising contacting Aβ fibrils with a sufficient amount of a pharmaceutical composition comprising a pharmaceutically acceptable carrier and an Aβ fibril binding compound selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or any combination thereof, wherein the interactions of the Aβ fibrils with a second binding molecule are inhibited.

17. The method of claim 16, wherein the Aβ fibril-binding compound is radiolabeled.

18. A method of preventing or alleviating the symptoms of an amyloid associated disease comprising contacting Aβ fibrils with a sufficient amount of a pharmaceutical composition comprising a pharmaceutically acceptable carrier and an Aβ fibril binding compound selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or any combination thereof, wherein the ion channel activity of the Aβ fibril decreases.

19. A composition comprising a compound bound to one or more Aβ fibrils, wherein the compound is selected from tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄ or BTA-EG₆, or any combination thereof.

20. A pharmaceutical composition comprising a compound suitable for treating a neuronal disease, wherein the compound is tannic acid, a derivative of tannic acid, nicotine, a pyrrolidine derivative of nicotine, a halogenated derivative of nicotine, an oligoethylene glycol derivative of nicotine, dopamine, curcumin, salicylic acid, norepinephrine, L-DOPA, N-methyl dopamine hydrochloride, BTA-EG₄, or BTA-EG₆, or any combination thereof, and wherein the compound inhibits or disrupts Aβ fibril interactions with cellular proteins.

21. The method of claim 20, wherein the neuronal disease is selected from the group consisting of Alzheimer's disease, Parkinson's disease, Huntington's disease, Down's Syndrome, cerebrovascular amyloidosis, Lewy body dementia, and spongiform encephalopathy.
